(19) United States
(12) Patent Application Publication
(10) Pub. No.: US 2023/0050396 A1
(43) Pub. Date: Feb. 16, 2023

(54) Collecting and Analyzing Swab Samples

(71) Applicants: MS Pen Technologies, Incorporated, Tulsa, OK (US); Board of Regents, The University of Texas System, Austin, TX (US)

(72) Inventors: Livia Schiavinato Eberlin, Austin, TX (US); Shannon D. Green, Tulsa, OK (US); Kyana Garza, Austin, TX (US); Sydney Cordano Povilaitis, Austin, TX (US); Michael Keating, Austin, TX (US); Jialing Zhang, Austin, TX (US); James Suliburk, Houston, TX (US)

(73) Assignees: MS Pen Technologies, Incorporated, Tulsa, OK (US); Board of Regents, The University of Texas System, Austin, TX (US)

(21) Appl. No.: 17/975,104

(22) Filed: Oct. 27, 2022

Related U.S. Application Data
(63) Continuation of application No. PCT/US2021/029819, filed on Apr. 29, 2021.
(60) Provisional application No. 63/017,387, filed on Apr. 29, 2020.
(60) Provisional application No. 63/088,724, filed on Oct. 7, 2020.

(51) Int. Cl. H01J 49/04; G01N 33/483; H01J 49/00; H01J 49/16

(52) U.S. Cl. CPC H01J 49/0409; G01N 33/4833; H01J 49/0027; H01J 49/167

(57) ABSTRACT

In a general aspect, a swab sample is analyzed, for example, to test for disease. In some examples, a swab head of a swab sample is inserted through an opening into an internal reservoir of a sampling device. The sampling device includes the opening, an inlet channel, an outlet channel, and the internal reservoir. The internal reservoir is in fluid communication with the inlet channel, the outlet channel, and the opening. A liquid solvent is supplied to the swab head in the internal reservoir via the inlet channel of the sampling device. The swab head is held in the liquid solvent for a period of time to form an analyte in the internal reservoir. The analyte is extracted from the internal reservoir via the outlet channel of the sampling device. The analyte is transferred to and processed by a mass spectrometer to obtain mass spectrometry data.

### CROSS REFERENCE TO RELATED APPLICATIONS

This application claims priority to U.S. Provisional Patent Application No. 63/017,387, filed Apr. 29, 2020, entitled “Collecting and Analyzing Swab Samples;” and U.S. Provisional Patent Application No. 63/088,724, filed Oct. 7, 2020, entitled “Collecting and Analyzing Swab Samples.” All of the above-referenced priority documents are incorporated herein by reference in their entirety.

### BACKGROUND

The following description relates to collecting and analyzing swab samples, for example, in the context of medical testing and diagnosis or another context.

Swab samples are often collected to diagnose diseases. Conventionally, a swab sample is collected by inserting a clean swab into a target region of a subject (e.g., into the subject's nose, throat, etc.) to collect a sample from the target region. The swab sample is then extracted from the subject and analyzed for the presence of disease markers.

As an example, a nasopharyngeal swab is often used to collect nasal secretions from the nasopharynx, and the nasopharyngeal swab sample may then be analyzed to test for whooping cough, diphtheria, influenza, diseases (e.g., SARS, MERS, and COVID-19) caused by the coronavirus family of viruses, and others. As another example, a throat swab is often used to collect saliva from the throat, and the throat swab sample may then be analyzed to test for bacteria or fungus that cause diseases such as strep throat, pneumonia, tonsillitis, and others.

### BRIEF DESCRIPTION OF THE DRAWINGS

FIG. 1 is a schematic diagram of an example swab sample analysis system.

FIG. 2 is a schematic diagram showing aspects of an example swab sample analysis system.

FIG. 3 is a schematic diagram showing aspects of an example sampling device.

FIG. 4 is a flowchart showing an example swab sample analysis process.

FIGS. 5A-5B are example mass spectra of two specimens.

FIG. 6 is a schematic diagram showing aspects of an example sampling device.

FIG. 7 is a flowchart showing an example swab sample analysis process.

FIGS. 8A-8C are schematic diagrams showing cross-sectional views of example tip portions.

FIG. 9 is a schematic diagram showing aspects of an example sampling cartridge.

FIG. 10 is a schematic diagram of an example swab sample analysis system.

FIGS. 11A-11B are schematic diagrams showing aspects of example swab sample analysis systems.

FIGS. 12A-12C are schematic diagrams showing aspects of an example ionization system.

FIGS. 13A-13B are schematic diagrams showing aspects of an example ionization source in an ionization system.

FIG. 13C is a photograph showing aspects of three example ionization sources.

FIG. 14A is a flow chart showing an example swab sample analysis process.

FIG. 14B is a flow chart showing an example tissue analysis process.

FIG. 15A is an example mass spectrum collected from a clinical nasal swab sample from a SARS-CoV-2 positive patient.

FIG. 15B is an example mass spectrum collected from a mouse brain tissue section.

FIGS. 16A-16C are plots showing molecular ion peaks and statistical weights associated with the molecular ion peaks in the example statistical models.

### DETAILED DESCRIPTION

In some aspects of what is described here, a swab sample analysis system includes a sampling device, a control system, and a mass spectrometer. The sampling device may be positioned on a sample surface (which may contain specimen collected from a patient) to receive a liquid solvent from the control system, to form an analyte (which may include at least a portion of the specimen collected on the sample surface), and to transfer the analyte to the mass spectrometer. The sampling device may be directly configured to receive a swab sample containing a specimen collected from a patient and to perform an on-site testing and screening. In certain examples, the swab sample analysis system may be formatted as a cartridge for high-throughput testing of multiple swab samples. The analyte received from the sampling device can be processed by the mass spectrometer. In some instances, disease markers in the swab samples can be identified, and the disease can be diagnosed (e.g., using a statistical classification model or another type of analysis).

In some examples, swab samples are collected (e.g., using nasal swab, nasopharyngeal swab, oropharyngeal swab, throat swab, etc.) to identify the presence of disease markers. The swab samples may include nasal swab samples (containing material from a nasal passage), nasopharyngeal swab samples (containing material from the nasopharynx), oropharyngeal swab samples (containing material from the oropharynx), throat swab samples (containing material from the throat) or other types of swab samples collected from a target region of a subject. The swab sample may then be processed by a mass spectrometer, and the mass spectrometry data may then be analyzed to detect disease markers.

In some examples, swab samples are tested to diagnose diseases, and the systems and techniques described here may be adapted to test for a wide range of diseases. For example, the systems and techniques described here may be adapted to test swab samples for bacterial infections, viral diseases, fungal infections, or other related pathogens and microorganisms. In some implementations, the systems and techniques described here may be adapted to test swab samples for whooping cough, diphtheria, influenza, diseases (e.g., SARS, MERS, and COVID-19) caused by the coronavirus family of viruses, strep throat, pneumonia, and tonsillitis.

In some of the examples described here, swab samples are tested to diagnose COVID-19 or another type of coronavirus disease. There is an urgent need for accelerated development and deployment of diagnostic tests for coronavirus diseases. Notably, the current need has arisen due to the global COVID-19 pandemic. Current diagnostic assays for coronavirus disease are largely based on the detection of the severe acute respiratory syndrome coronavirus 2 (SARS-CoV-2) virus via quantitative polymerase chain reaction (qPCR) analysis. While PCR assays are powerful and highly sensitive, clinical laboratories have been facing immense challenges in keeping up with the current demands due to limited availability of PCR test reagents, failing PCR instrumentation that are being overrun beyond their capabilities, and the low throughput of PCR analysis. Serological tests targeting host antibodies are being deployed for the coronavirus disease diagnosis and are yielding highly promising results, yet their limited sensitivity at early infection stages represents a potential challenge for patient screening.

The current global pandemic illustrates the need for improved systems and methodologies for collecting and analyzing swab samples. As we have seen, the need to rapidly diagnose novel diseases can arise in a matter of weeks or months, which demonstrates a further advantage in developing systems and methodologies that can be adapted to diagnose other diseases (e.g., currently known diseases, and other diseases that may arise in the future).

In some implementations, the methods and systems disclosed here may provide technical advantages and improvements relative to conventional techniques. In some instances, the methods and systems described here use a common and inexpensive laboratory solvent without using primers and/or amplification. In some instances, the methods and systems described here can provide high molecular specificity and sensitivity for detecting diseases. In some instances, the methods and systems described here can provide high sensitivity and selectivity for untargeted chemical analysis of molecules such as metabolites, lipids, and proteins from pathogen/host with minimal sample preparation requirements. Additionally, simplified operational steps and system design may be utilized without requiring experienced professionals to perform such analysis and diagnosis, which allows the use of such systems and methods as a rapid screening tool and deployed publicly without being limited by the capacity of healthcare workforce. In some instances, the methods and systems described here can potentially minimize risks for healthcare professionals that are at this time being exposed to the aerosols formed during the swabbing procedure currently employed in the clinic for specimen collection. In some instances, the methods and systems described here can identify clinically relevant prognostic biomarkers for diseases to predict clinical courses to enable proactively planning for healthcare resources. In some cases, a combination of these and potentially other advantages and improvements may be obtained.

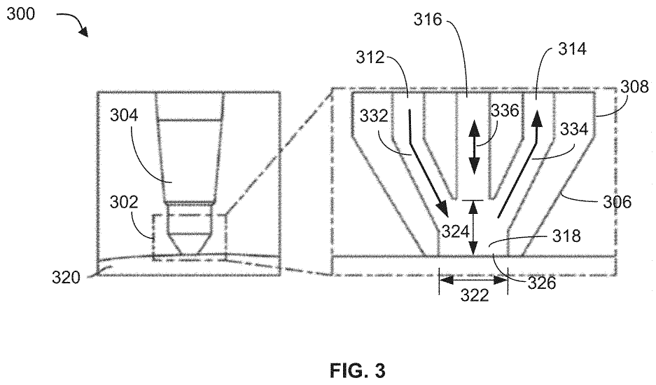

In some implementations, a sampling device includes a tip portion and a housing. In some implementations, the tip portion, e.g., the tip portion as shown in FIGS. 3, 6 and 8, may include a mandrel end and a cylindrical end. For example, the mandrel end in a tapered cylindrical shape may be used for contacting a sample surface, which may or may not contain the disease marker (e.g., a marker of SARS-CoV-2 virus, or otherwise). For example, the cylindrical end may be used to engage with a receiving end of the housing. In some implementations, the tip portion may include three internal channels creating three internal pathways and an internal reservoir. In some examples, the tip portion may include a liquid supply channel (e.g., the liquid supply channel), a liquid extraction channel (e.g., the liquid extraction channel), and a gas channel (e.g., the gas channel). In some implementations, the liquid supply and extraction channels may be configured to provide fluid communication with the control system and the mass spectrometer.

In some implementations, the liquid supply channel is configured for receiving a liquid solvent from an external container, for guiding the liquid solvent to the internal reservoir at the tip portion, where the liquid solvent may be in direct contact with the swab sample inserted into the internal reservoir through an opening, and for filling up at least a portion of the internal reservoir with the liquid solvent. In some implementations, the liquid extraction channel is configured for obtaining an analyte from the internal reservoir by extracting at least a portion of the liquid solvent carrying suspended cells and/or extracted molecules from the cells, and for guiding the analyte to the transfer tube.

In some implementations, a fixed volume of liquid solvent is communicated into the tip portion. The fixed volume of fluid can be retained within the internal reservoir while in direct contact with the sample surface for a controlled amount of time, to form an analyte containing molecules from the sample surface. The analyte may then be extracted (e.g., as a single, discrete droplet of fluid) from the internal reservoir through the liquid extraction channel for analysis. In some instances, the analyte is produced by the probe in a non-destructive manner that does not damage the sample surface. For instance, the probe may extract the analyte from a tissue site or tissue sample without causing any detectable damage or destruction to the tissue.

In some implementations, a swab sample analysis system includes an ionization system which can receive the analyte from the sampling device via the transfer tube and can be used to ionize the analyte to produce ions of the molecules in the suspended cells and/or extracted molecules from the cells. In some instances, the ionization system contains an electrospray ionization source under vacuum that can be directly integrated between a sampling device and a mass spectrometer. In some instances, the vacuum created within the ionization system, for example in a chamber, can provide a driving force required to extract/transport an analyte from the sampling device to the ionization system. In some instances, the ionization system can be used to improve the ionization efficiency, sensitivity, and reproducibility of the swab sample analysis system.

In some implementations, a swab sample analysis system includes a mass spectrometer that produces mass spectral data which can include molecular profiles for differentiation and identification of disease markers. In some implementations, the swab sample analysis system includes a statistical classification model to provide separations of strains according to the molecular profiles. In some instances, fa statistical classification model, e.g., a multi-level LASSO model, may allow discrimination of disease markers (e.g., markers for SARS-CoV-2 virus and its various strains, etc.).

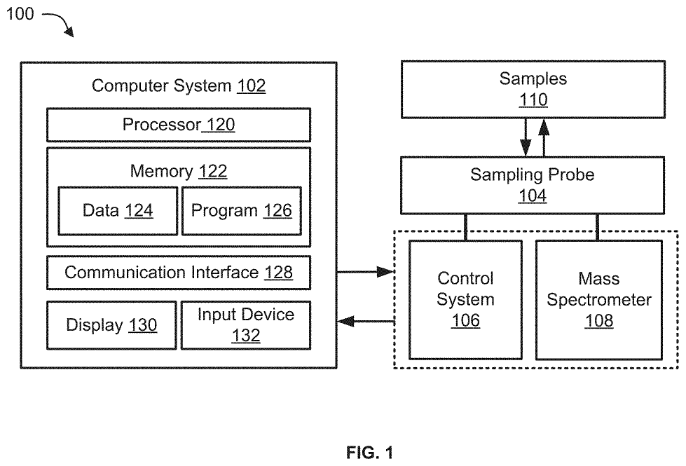

FIG. 1 is a schematic diagram of an example swab sample analysis system 100. As shown in FIG. 1, the example system 100 includes a computer system 102, a sampling device 104, a control system 106, and a mass spectrometer 108. In some implementations, the example system 100 may be used for qualitatively and quantitatively identification and classification of specific markers of disease (e.g., the SARS-CoV-2 virus and its various strains, or other types of diseases). In some implementations, the system 100 is used in a laboratory environment to evaluate biological samples collected using swabs, or in another manner. In some instances, the system 100 can be used for on-site testing and screening. In some examples, the example system 100 may include additional or different components, and the components may be arranged as shown or in another manner.

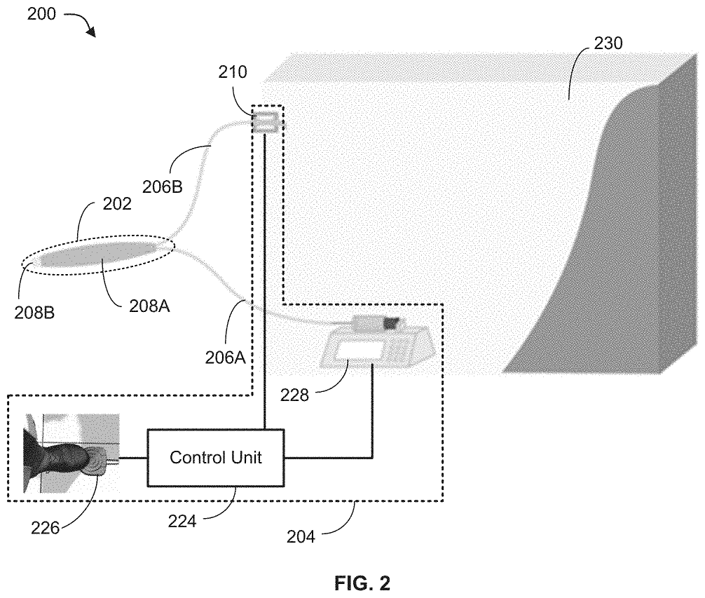

In the example shown in FIG. 1, the computer system 102 includes a processor 120, memory 122, a communication interface 128, a display device 130, and an input device 132. In some implementations, the computer system 102 may include additional components, such as, for example, input/output controllers, communication links, power, etc. In some instances, the computer system 102 may be configured to control operational parameters of and to receive data from the control system 106, and the mass spectrometer 108. The computer system 102 can be used to control the control system 106 to deliver liquid solvents to the sampling device 104; and to control the extraction of analytes containing suspended cells and molecules from the sampling device 102. In some implementations, the computer system 102 may be used to implement one or more aspects of the systems and processes described with respect to FIGS. 2, and 3, or to perform another type of operation. In some implementations, the computer system 102 includes a separate control unit associated with and providing specific control functions to the control system 106. In some instances, the control unit may be implemented as the example control unit 224 in FIG. 2, or in another manner.

In some implementations, the computer system 102 may include a single computing device, or multiple computers that operate in proximity to, or remote from, the rest of the example system 100 (e.g., the control system 106, and the mass spectrometer 108). In some implementations, the computer system 102 may communicate with the rest of the example system 100 via the communication interface 128 through a communication network, e.g., a local area network (LAN), a wide area network (WAN), an inter-network (e.g., the Internet), a network comprising a satellite link, and peer-to-peer networks (e.g., ad hoc peer-to-peer networks).

In some implementations, the sampling device 104 may be configured to communicate fluids with the control system 106 and the mass spectrometer 108 via transfer tubes. In some instances, the sampling device 104 may receive liquid solvent from the control system 106, guide the liquid solvent to a sample 110 with specimen collected from a patient, obtain an analyte by extracting at least a portion of the liquid solvent from the sample 110, and deliver the analyte containing suspended viruses/host cells and molecules extracted from the viruses/host cells to the mass spectrometer 108. In some implementations, the sampling device 104 may include a tip portion, which may include multiple internal liquid/gas channels and an internal reservoir, e.g., the channels 312, 314, 316 and the internal reservoir 318 as shown in FIG. 3. In some implementations, the sampling device 104 may be composed of materials, such as synthetic polymers that are biologically compatible and resistant to chemicals used. In some examples, the sampling device 104 may be implemented as the sampling devices 202, 300, 602, and 800 as shown in FIGS. 2-3, 6 and 8, or in another manner.

The control system 106 controls the movement of fluid in the swab sample analysis system 100. In some implementations, the control system 106 may include a mechanical pumping system and one or more mechanical valves. In some instances, the mechanical pumping system contains a mechanical pump that is controlled by the computer system 102. For example, the mechanical pumping system may be implemented as the mechanical pumping system 228 as shown in FIG. 2 or in another manner. In some instances, the control system 106 may provide high-precision, microfluidic dispensation of the liquid solvent to the internal reservoir of the sampling device 104. In some instances, a control unit of the control system 106 (e.g., the control unit 224 in FIG. 2) may be configured to trigger and control a sampling process by controlling the mechanical pumping system and the one or more mechanical valves. In some instances, the control unit of the control system 106 may be configured to simultaneously trigger a data collection process by the mass spectrometer 108. In some implementations, the liquid solvent may include sterile water, ethanol, methanol, acetonitrile, dimethylformamide, acetone, isopropyl alcohol, or a combination.

In some implementations, an analyte may be received by the mass spectrometer 108. In some instances, the analyte may include the liquid solvent and one or more of the following: pathogen molecules, molecules from cells of the host, or molecules in secretions produced by the cells of the host. In some implementations, the analyte may be extracted from the sampling device 202 by creating a low pressure in the mass spectrometer 108. For example, the low pressure can be created by a vacuum pump attached to the mass spectrometer 108. In some implementations, prior to the mass spectrometer, the analyte may be collected and delivered to an ion optic system. In some instances, the ion optic system may be configured to filter neutral species in the analyte, to allow ions passing through, and to eliminate contamination of the mass spectrometer 108. In some implementations, the mass spectrometer 108 may include a mass selector and a mass analyzer, which are configured to separate and identify the ionization products in the ionized analyte according to their mass-to-charge (m/z) ratio. In some implementations, the mass spectrometer 108 may output a set of mass spectra (e.g., intensity of the ionized product vs. the m/z ratio) to the computer system 102, which may be stored in the memory 122, analyzed by running a program 126 and results may be further displayed on the display 130. In some implementations, the mass spectrometer 108 may be implemented as the mass spectrometer 230 as shown in FIG. 2 or in different manner.

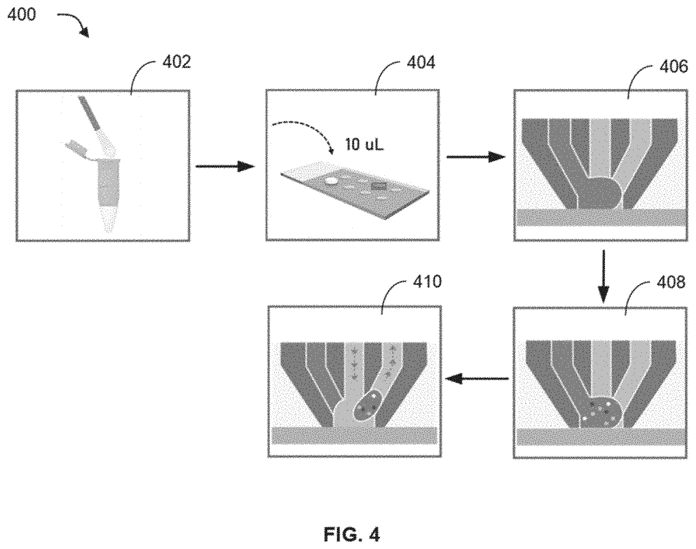

In some implementations, some of the processes and logic flows described in this specification can be automatically performed by one or more programmable processors, e.g. processor 120, executing one or more computer programs to perform actions by operating on input data and generating output. For example, the processor 120 can run the programs 126 by executing or interpreting scripts, functions, executables, or other modules contained in the programs 126. In some implementations, the processor 120 may perform one or more of the operations described, for example, with respect to FIGS. 4, 7, and 13.

In some implementations, the processor 120 can include various kinds of apparatus, devices, and machines for processing data, including, by way of example, a programmable data processor, a system on a chip, or multiple ones, or combinations, of the foregoing. In certain instances, the processor 120 may include special purpose logic circuitry, e.g., an Arduino board, an FPGA (field programmable gate array), an ASIC (application specific integrated circuit), or a Graphics Processing Unit (GPU) for running the deep learning algorithms. In some instances, the processor 120 may include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. In some examples, the processor 120 may include, by way of example, both general and special purpose microprocessors, and processors of any kind of digital computer.

In some implementations, the processor 120 may include both general and special purpose microprocessors, and processors of any kind of quantum or classic computer. Generally, a processor 120 receives instructions and data from a read-only memory or a random-access memory or both, e.g. memory 122. In some implementations, the memory 122 may include all forms of non-volatile memory, media and memory devices, including by way of example semiconductor memory devices (e.g., EPROM, EEPROM, flash memory devices, and others), magnetic disks (e.g., internal hard disks, removable disks, and others), magneto optical disks, and CD ROM and DVD-ROM disks. In some cases, the processor 120 and the memory 122 can be supplemented by, or incorporated in, special purpose logic circuitry.

In some implementations, the data 124 stored in the memory 122 may include, operational parameters, a standard reference database and output data. In some instances, the standard reference database includes a mass spectral reference library, which may be used for identification of disease markers. In some implementations, the programs 126 can include software applications, scripts, programs, functions, executables, or other modules that are interpreted or executed by the processor 120. In some implementations, the programs 126 may include machine-readable instructions for performing deep learning algorithms. In some instances, the programs 126 may include machine-readable instructions for delivering the liquid solvent to the sampling device, and collecting the analyte from the sampling device. In some instances, the programs 126 may obtain input data from the memory 122, from another local source, or from one or more remote sources (e.g., via a communication link). In some instances, the programs 126 may generate output data and store the output data in the memory 122, in another local medium, or in one or more remote devices (e.g., by sending the output data via the communication network 106). In some examples, the programs 126 (also known as, software, software applications, scripts, or codes) can be written in any form of programming language, including compiled or interpreted languages, declarative or procedural languages. In some implementations, the programs 126 can be deployed to be executed on the computer system 102.

In some implementations, the communication interface 128 may be connected to a communication network, which may include any type of communication channel, connector, data communication network, or other link. In some instances, the communication interface 128 may provide communication with other systems or devices. In some instances, the communication interface 128 may include a wireless communication interface that provides wireless communication under various wireless protocols, such as, for example, Bluetooth, Wi-Fi, Near Field Communication (NFC), GSM voice calls, SMS, EMS, or MMS messaging, wireless standards (e.g., CDMA, TDMA, PDC, WCDMA, CDMA2000, GPRS) among others. In some examples, such communication may occur, for example, through a radio-frequency transceiver or another type of component. In some instances, the communication interface 128 may include a wired communication interface (e.g., USB, Ethernet) that can be connected to one or more input/output devices, such as, for example, a keyboard, a pointing device, a scanner, or a networking device such as a switch or router, for example, through a network adapter.

In some implementations, the communication interface 128 can be coupled to input devices and output devices (e.g., the display device 130, the input device 132, or other devices) and to one or more communication links. In the example shown, the display device 130 is a computer monitor for displaying information to the user or another type of display device. In some implementations, the input device 132 is a keyboard, a pointing device (e.g., a mouse, a trackball, a tablet, and a touch sensitive screen), or another type of input device, by which the user can provide input to the computer system 102. In some examples, the computer system 102 may include other types of input devices, output devices, or both (e.g., mouse, touchpad, touchscreen, microphone, motion sensors, etc.). The input devices and output devices can receive and transmit data in analog or digital form over communication links such as a wired link (e.g., USB, etc.), a wireless link (e.g., Bluetooth, NFC, infrared, radio frequency, or others), or another type of link.

In some implementations, other kinds of devices may be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. For example, the sampling device 104 may contain a control element (e.g., button, pedal, etc.) which may be used as a controller to initiate, interrupt, restart, or terminate a detection process (e.g., the pedal 226 as shown in FIG. 2). In some instances, a graphic user interface (GUI) may be used to provide interactions between a user and the swab sample analysis system 100. In certain instances, the GUI may be communicably coupled to the computer system 102. For example, when the control system 106 is activated (e.g., by pushing on the pedal 226 in FIG. 2), the GUI can initiate an analysis process in the mass spectrometer 108. For example, when the analysis process is completed, the GUI can output a report with analysis results.

FIG. 2 is a schematic diagram showing aspects of an example swab sample analysis system 200. In the example shown in FIG. 2, the example system 200 includes a sampling device 202, a control system 204, and a mass spectrometer 230. As shown in FIG. 2, the sampling device 202 is coupled between the control system 204 and the mass spectrometer 230 through transfer tubes 206A, 206B. In some examples, the example system 200 may include additional or different components, and the components may be arranged as shown or in another manner.

In the example shown in FIG. 2, the sampling device 202 includes a housing 208A and a tip portion 208B. In some implementations, the housing 208A may provide a grip for being used as a handheld sampling device. In some implementations, the housing 208A may include a control element, e.g., a trigger or button. For example, the control element may be used to control the liquid solvent transferring through the sampling device 202. In some instances, the control element may be separated from the housing 208A, e.g., configured as a foot pedal. For another example, the control element may be coupled to a mechanism which may be used to eject the tip portion 208B. In some implementations, the sampling device 202 may be composed of materials, such as synthetic polymers that are biologically compatible and resistant to chemicals used in the measurement. For example, the materials for the sampling device 202 may be compatible with a variety of liquid solvent (e.g., polar or non-polar) that is used for extracting and carrying an analyte to the mass spectrometer 230. In some examples, the synthetic polymers that may be used for fabricating the sampling device 202 may include Polydimethylsiloxane (PDMS), or Polytetrafluoroethylene (PTFE). In some implementations, the tip portion 208B may use the same material as the housing 208A, different materials or different compositions. In some instances, geometries of the sampling device 202 may be designed to fit into a nose or a throat of a patient to directly perform sample secretions in vivo for immediate analysis/diagnosis. In some instances, the sampling device 202 may be directly coupled to an external container, e.g., a vial and used as a sample collection device for further analysis in a specialized lab.

In some implementations, the sampling device 202 may be manufactured using a 3D printing process, a machining process or another process. In some implementations, the housing 208A of the sampling device 202 may include two internal channels which are fluidically coupled with respective transfer tubes 206A, 206B and respective channels in the tip portion 208B. In some implementations, the transfer tubes 206A, 206B are configured for supplying a liquid solvent from the control system 204 to the tip portion 208B and to obtain an analyte by collecting at least a portion of the liquid solvent with suspended viruses/host cells and extracted molecules from the tip portion 208B. The sampling device 202 may also include a gas channel (e.g., an open port that receives air from the surrounding atmosphere) that allows liquid to be flushed from the sampling device 202, for example, between uses or at other instances.

In some implementations, the tip portion 208B may be detachable from the housing 208A, which can be disposed and replaced if contaminated, e.g., after a certain number (e.g., one or more) of regular uses or when switching between different samples. In some cases, the tip portion 208B may include internal channels that are fluidically coupled to the respective channels in the housing 208A and further to the respective transfer tubes 206A, 206B. In some implementations, the tip portion 208B may be integrated with the housing 208A as a monolithic structure. In some implementations, the tip portion 208B may be implemented as the tip portion 302 as shown in FIG. 3 or in another manner.

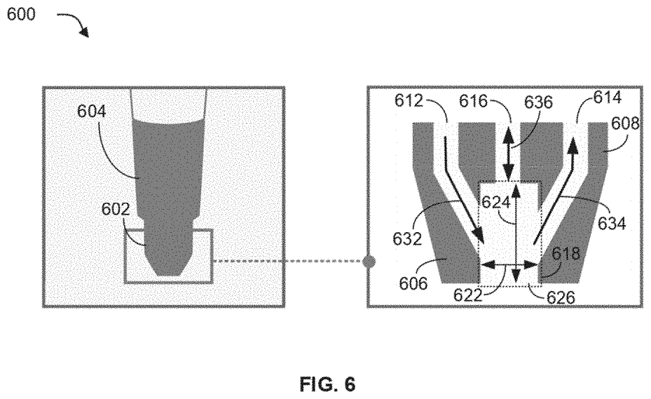

In some implementations, the control system 204 may include an external container and a mechanical pumping system 228. In some instances, the mechanical pumping system 228 may contain one or more mechanical pumps. In some instances, the one or more mechanical pumps may be programmable. In certain examples, the one or more mechanical pumps may be controlled by a computer system, e.g., the computer system 102 in FIG. 1. In some implementations, a mechanical pump may be a syringe pump, a peristatic pump or other type of pump, which can provide high-precision, microfluidic dispensation of the liquid solvent to the tip portion 208B, e.g., the internal reservoir 318, 618 of the tip portion 302, 602 as shown in FIGS. 3 and 6. In some implementations, each of the one or more mechanical pumps may be equipped with separate solvent containers containing different types of liquid solvents. In the example shown in FIG. 2, the liquid solvent in a container (e.g., syringe) can be delivered to the sampling device 202 through a first transfer tube 206A. In some implementations, the control system 204 may supply a controlled volume of liquid solvent to the sampling device 202 at a controlled flow rate according to the design of the tip portion 208B, e.g., the volume of the internal reservoirs as shown in FIGS. 3, 6 and 8.

As shown in FIG. 2, the control system 204 further includes one or more valves on the transfer tubes 206A, 206B. In some implementations, each of the one or more valves is configured to control a fluidic flow (e.g., start or stop a fluidic flow) in respective transfer tubes. In some implementations, each of the one or more valves may be mechanically activated and electrically controlled by a computer system, e.g., the computer system 102 as shown in FIG. 1. In some examples, the one or more valves 210 may include a pinch valve, a squeeze valve, other type of valve, or a combination. In some instances, the valve 210 on a second transfer tube 206B is a high-speed actuated pinch valve for controlling aspiration and extraction of the analyte to the ionization system 220. In some instances, the control system 204 is communicably coupled with a control unit 224. In some instances, the control unit 224 may include an Arduino board to control motions of the mechanical pumping system 228 and the one or more valves 210. As shown, the control unit 224 can be activated by pushing a pedal 226 and deactivated by releasing the pedal 226. In some instances, when activated, the control unit 224 may also initiate a data collection process performed by the mass spectrometer 230.

In some implementations, the transfer tubes 206A, 206B may have a length in the range of approximately half a meter to one or more meters (e.g., a length in the range of approximately 0.5 m to 1.5 m, or in another range) to allow free handheld use of the sampling device 202 by an operator without geometrical or spatial constraints.

In some implementations, the analyte may be collected and delivered to an ion optic system prior to the mass spectrometer 230. In some instances, the ion optic system may be configured to filter neutral species in the analyte, to allow ions passing through, and to eliminate contamination to the mass spectrometer 230.

In some implementations, the mass spectrometer 230 may include a mass selector and a mass analyzer. In some implementations, the mass selector may separate charged biomolecules according to their mass-to-charge (m/z) ratio based on dynamics of charged particles in electric and magnetic field in vacuum. The mass selector may include a set of magnets providing a magnetic field, which the charged molecules travel through. The mass selector may use the magnetic field to alter the path of the charged molecules so that they can be separated according to their charges and mass. In some examples, the mass analyzer may output a set of mass spectra (or mass spectrometry data in another format) for data analysis.

In some implementations, when an analysis is completed by the mass spectrometer 230, the mass spectrometer 230 may produce a report with analysis results, for example, a strain type of the SARS-CoV-2 virus. In some instances, mass spectrometry data produced by the mass spectrometer 230 may be analyzed, and the results of the analysis can be used to determine an appropriate treatment for a patient.

FIG. 3 is a schematic diagram showing aspects of a sampling device 300, which can be used in a swab sample analysis system such as, for example, those shown in FIGS. 1 and 2. As shown in FIG. 3, the sampling device 300 includes a tip portion 302 and a housing 304. The example tip portion 302 includes a mandrel end 306 in a tapered cylindrical shape which is used for contacting a sample surface 320, and a cylindrical end 308 engaged with a receiving end of the housing 304. In some implementations, the cylindrical end 308 seals with the receiving end of the housing 304 (e.g., making an air-tight seal). In some examples, the tip portion 302 may include additional or different components, and the components may be arranged as shown or in another manner.

As shown in a cross-sectional view of the tip portion 302 in FIG. 3, the tip portion 302 includes three distinct internal channels, including a liquid supply channel 312, a liquid extraction channel 314, and a gas channel 316. In some implementations, the three internal channels 312, 314, 316 are aligned with respective internal channels (not shown) in the receiving end of the housing 304 to provide fluidic communication with transfer tubes. In some instances, the transfer tubes may be implemented as the transfer tubes 206A, 206B as shown in FIG. 2 or in another manner. In some implementations, the three internal channels 312, 314, 316 may be directly coupled with transfer tubes that extends through the housing 304 from the end opposite to the receiving end to the receiving end of the housing 304 or may be coupled with the transfer tubes in other manner to allow liquid and gas flow.

In some implementations, the housing 304 is configured to provide fluidic communication with a control system and a mass spectrometer through respective transfer tubes, e.g., the transfer tubes 206A, 206B as shown in FIG. 2. In some implementations, the housing 304 and the tip portion 302 may be composed of biologically compatible synthetic polymers. In some implementations, the housing 304 and the tip portion 302 may be fabricated using a 3D printing process, a machining process or another type of fabrication process.

In some implementations, the sample surface 320 may be a surface of a solid substrate. For example, the sample surface 320 may be a glass slide, a 24-well Polytetrafluoroethylene (PTFE)-coated glass slide, a petri dish, or an agar plate. In some implementations, the sample surface 320 contacts the mandrel end 306 of the tip portion 302 to form a seal (e.g., a liquid-tight seal), in order to prevent leakage of the liquid solvent from the internal reservoir 318. In some implementations, the sample surface 320 may contain specimen collected from a patient. For example, the specimen may be collected using a swab, e.g., nasal, nasopharyngeal, oropharyngeal, or throat swab, extracted into a vial of solvent, and transferred onto the sample surface 320. In some instances, the sampling device is used to collect an analyte in order to determine whether the sample surface 320 does or does not contain a disease marker (e.g., SARS-CoV-2 virus or another).

In some aspects of operation, the liquid supply channel 312 receives the liquid solvent from an external container, guides the liquid solvent to the internal reservoir 318 at the tip portion 302, where the liquid solvent may be in direct contact with the sample surface 320 via an opening 326, and fills at least a portion of the internal reservoir 318 with the liquid solvent. The liquid supply channel 312 may provide a first internal pathway 332 in the tip portion. In some implementations, the liquid solvent may be received from the external container as a part of a control system, e.g., the syringe pump as shown in FIG. 2 or another type of mechanical pumping system.

In some implementations, the internal reservoir 318 may have a cylindrical shape and be coupled to the liquid supply channels 312. In certain examples, the liquid solvent received from the liquid supply channel 312 in the internal reservoir 318 makes direct contact with the sample surface 320. In some instances, at least a portion of a specimen collected from a patient may be suspended such that molecules from cells in a virus may be extracted into the liquid solvent. In some implementations, the diameter 322 of the internal reservoir 318 is determined by, for example, the size of the sample surface 320 and the amount of the virus on the sample surface 320. In some instances, the diameter 322 and height 324 of the internal reservoir 318 may determine the volume of the liquid solvent exposed to the sample surface 320 and performance aspects of the chemical measurement system, for example a spatial resolution, limit of detection, and accuracy. In some instances, the diameter of the internal reservoir 318 of the tip portion 302 may be in a range of 1.5-5.0 mm. For example, when the diameter 322 of the internal reservoir is 2.77 mm and the height 324 of the internal reservoir 318 is 1.7 mm, the volume of a liquid solvent that is contained in the internal reservoir 318 is 10 microliter (4). For another example, when the diameter of the internal reservoir 318 is 1.5 mm and the height 324 is 2.5 mm, the volume of a liquid solvent that is contained in the internal reservoir 318 is 4.4 μL. The internal reservoir 318 may have a different shape, aspect ratio, size or dimension.

In some instances, the liquid extraction channel 314 provides a second, distinct internal pathway 334 in the tip portion 302. In some aspects of operation, the liquid extraction channel 314 obtains an analyte which includes at least a portion of the liquid solvent carrying the suspended cells or the extracted molecules from the internal reservoir 318, extracts and guides the analyte to the transfer tube that is coupled to a mass spectrometer. In some implementations, the analyte from the internal reservoir 318 may be extracted by a vacuum pump coupled to the mass spectrometer (e.g., the mass spectrometer 230 as shown in FIG. 2). In some implementations, a low pressure created on one end of the transfer tube may facilitate liquid aspiration to drive the analyte from the internal reservoir 318 to the mass spectrometer through the liquid extraction channel 314.

In some implementations, the gas channel 316 provides a third, distinct internal pathway 336 in the tip portion 302. In some instances, the gas channel 316 is configured for preventing collapse of the sampling device, transfer tubes and the control system during the extraction. In some instances, the gas channel 316 is open to atmosphere (e.g., air). In some instances, diameters of the liquid supply channel 312, the liquid extraction channel 314 and the gas channel 316 may be equal to 0.8 mm. Gas from the gas channel 316 may be used to push the liquid out of the liquid extraction channel 314 to the mass spectrometer.

FIG. 4 is a flow chart showing an example swab sample analysis process 400. In some implementations, aspects of the example process 400 may be automated for detection and identification of SARS-CoV-2 virus or another disease marker. The example process 400 can be performed, for example, by a swab sample analysis system. For instance, operations in the example process 400 may be performed by the swab sample analysis system shown in FIGS. 1-3 or another type of swab sample analysis system with additional or different components. The example process 400 may include additional or different operations, including operations performed by additional or different components, and the operations may be performed in the order shown or in another order. In some cases, operations in the example process 400 can be combined, iterated or otherwise repeated or performed in another manner.

At 402, a specimen from a swab head is extracted into a liquid solvent. The specimen can include a swab sample collected from a patient. In some instances, the specimen may include material collected using a nasal swab, nasopharyngeal swab, oropharyngeal swab, throat swab, or another type of swab. In some instances, the swab head is placed into a sterile viral transportation mediate tube before being tested. In some instances, other type of specimens with higher viral load may be collected and tested, e.g., sputum or saliva.

In some instances, the liquid solvent may contain a mixture of chloroform and methanol (e.g., 2:1, v/v) or another mixture with different types of solvents. In some instances, the liquid solvent may be used to extract various fatty acids including palmitic acid, oleic acid, and linoleic acid, and glycerophospholipids in the virus envelope and host cellular membranes including phosphatidylethanolamine (PE), phosphatidylserine (PS), phosphatidylinositol (PI) and phosphatidylglycerol (PG) species from the specimen. In some instances, the extraction may be performed in a vial, and after extraction, an analyte is formed in the vial for analysis. In some instances, one or more sample pre-processing steps, e.g., pre-concentration, may be further performed after the extraction. In some instances, liquid and protein standards with known concentrations are also prepared for analysis. In some instances, specimens from healthy individuals may be also collected as control samples for comparison. In some examples, SARS-CoV-2 virus (strain 2019nCoV/USAWA1/2020) may be heat-inactivated and obtained from the American Type Culture Collection (ATCC) and the Biodefense and Emerging Infections Research Resources Repository (BEI Resources) under research exemption and biosafety committee approvals. In some instances, non-invasive respiratory specimens may be obtained from research institutions or biorepository companies. In some instances, positive and negative control samples may be developed using lipid standards identified as markers for the SARS-CoV-2 virus and control samples, respectively, and spiked into a synthetic nasopharyngeal (NP) matrix or in another manner. In some instances, inactivated SARS-CoV-2 viruses may be spiked into clinical matrix for determining the Assay Limit of Detection using qPCR as a gold standard. In some instances, the inactivated SARS-CoV-2 viruses spiked into clinical matrix may be also used for determining analytical sensitivity. In some instances, the detection targets characterized by mass spectrometer may be spiked into clinical matrix as control samples.

At 404, the specimen is transferred onto a sample substrate. In some examples, the suspended viruses/host cells and the extracted molecules from the specimen may be deposited by dropping a fixed volume of the specimen in the liquid solvent on the sample surface followed by drying in air at room temperature. In some instances, the sample substrate is a 24-well Polytetrafluoroethylene (PTFE)-coated glass slide or another type of substrate or container.

At 406, a liquid solvent is supplied. In some implementations, the liquid solvent may be supplied to the sample surface to extract (e.g., dissolve or otherwise mix with) the specimen. In some implementations, the sample surface may be implemented as the sample surface 320 as shown in FIG. 3 or in another manner. In some implementations, the liquid solvent is supplied to the sample surface with controlled volume and flow rate. In some implementations, a control system (e.g., the control system 204 as shown in FIG. 2) may be used to supply the liquid solvent to a sampling device with an internal reservoir at an opening (e.g., the sampling device 300 as shown in FIG. 3), where the liquid solvent may be in direct contact with the sample surface. In some implementations, the control system may be controlled by a control unit (e.g., the control unit 224 as shown in FIG. 2). In certain examples, the liquid solvent may be supplied to the internal reservoir of the sampling device.

At 408, an analyte is formed. In some implementations, the liquid solvent after being delivered to the internal reservoir of the sampling device may interact with at least a portion of the specimen at the sample surface causing the molecules to be extracted and dissolved in (or otherwise mix with) the liquid solvent contained in the internal reservoir. In some implementations, the liquid solvent may be allowed to interact with the viruses/host cells and the extracted molecules on the sample surface for a certain time period. In some implementations, the time period may be determined by one or more of the type of the liquid solvent (e.g., polar or non-polar), temperature, and solubility of the species in a solvent.

At 410, the analyte is extracted. In some implementations, the analyte may include the liquid solvent mixed with the specimen. In some implementations, extraction of the analyte from the sample surface may be performed by applying a pressure on one end of a transfer tube coupled to the sampling device. In some instances, the pressure may be lower than the atmospheric pressure. In some implementations, the analyte may be extracted into and analyzed by a mass spectrometer. In certain examples, prior to being analyzed by the mass spectrometer, the analyte may be ionized using an electrospray ionization or another manner. In some instances, the mass spectrometer of the swab sample analysis system include an Orbitrap QE Mass Spectrometer operating under a negative ion mode with a resolving power: 120,000 and a mass accuracy <5 ppm. In some instances, the mass spectrometer may be operated under a positive ion mode to increase chemical coverage, e.g., SARS-CoV-2 nucleocapsid proteins, and predictive strength. In certain examples, other solvent systems to extract a wider range of metabolites and proteins may be used in combination with the mass spectrometer operating under different ion mode.

In some implementations, the mass spectral data may be imported into a commercial statistical analysis software, e.g., RStudio. In some instances, after importing, the mass spectrometry data may be binned to m/z=0.01, and normalized to a total ion current. In some instances, background including molecular features originating from the liquid solvent may be subtracted from the mass spectrometry data. In some implementations, a principle component analysis is performed on the processed mass spectral data and results including classification can be returned. For example, a prcomp function in RStudio is used. In some implementations, limit of detection may be determined by performing the example process 400 on various heat-inactivated SARS-CoV-2 virus samples (e.g., 500 viral copies) and/or titer information from clinical samples. In some instances, molecular features in mass spectra obtained from samples collected from coronavirus-positive patients may be associated with their clinical outcome, including asymptomatic, mild (recovery at home), moderate (non-ICU hospitalization), and severe (ICU hospitalization) clinical course. In some instances, robust predictive models can be constructed to accurately diagnose the coronavirus disease and other related pathogens, and to identify novel predictive markers of the disease and of host response. Reproducibility, accuracy, analytical reactivity, and analytical specificity for high priority pathogens and organisms spiked into controls, and for clinical samples may be evaluated.

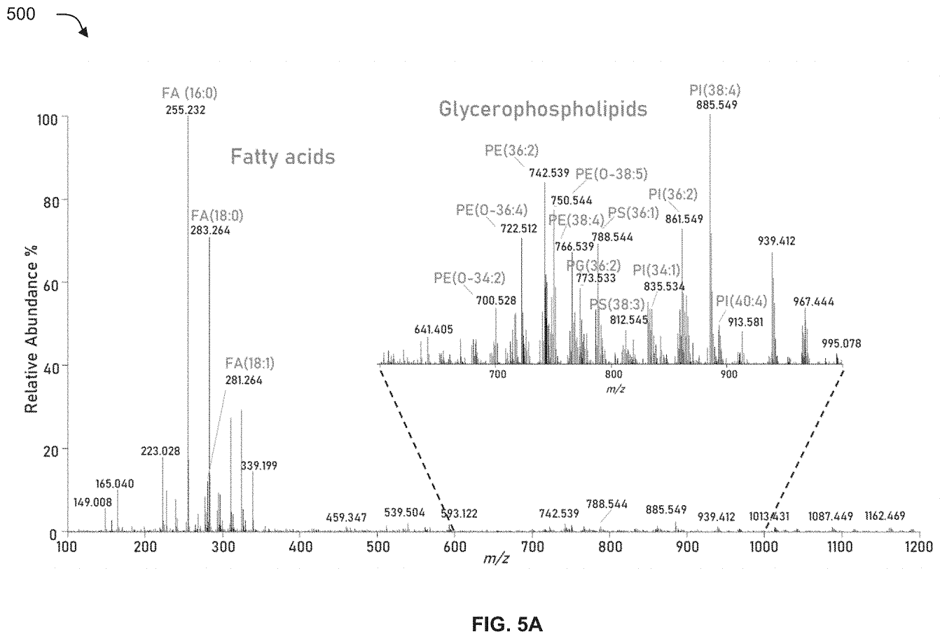

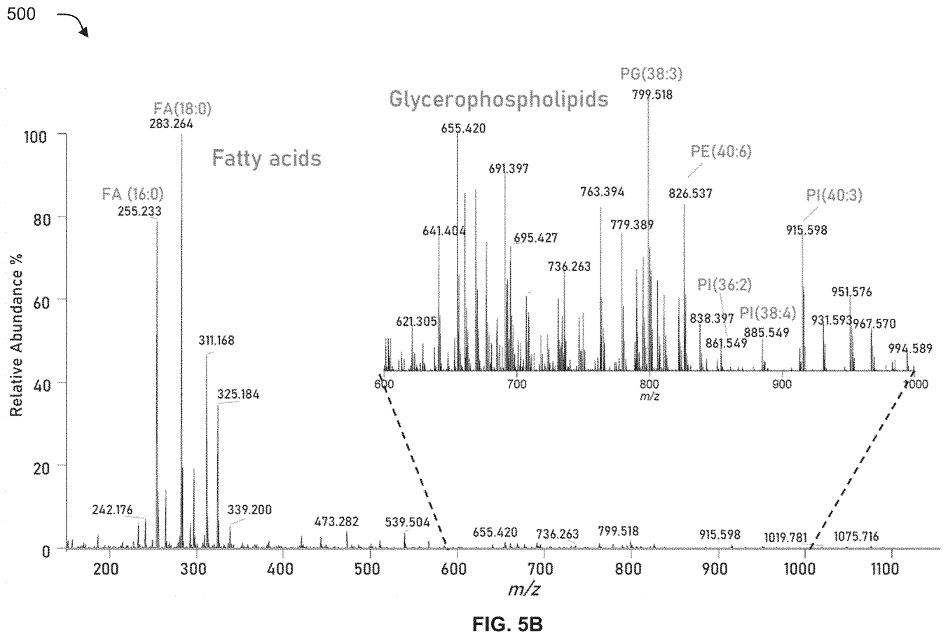

FIGS. 5A-5B are example mass spectra 500 of two specimens. As show in FIGS. 5A-5B, the example mass spectra is obtained on a heat-inactivated SARS-CoV-2 virus sample (strain 2019nCoV/USAWA1/2020) as shown in FIG. 5A and on a nasal swab extract from a patient as shown in FIG. 5B. In the example shown in FIGS. 5A-5B, mass spectra may be collected by a swab sample analysis system. In some implementations, a swab sample analysis system may be used to detect and identify the SARS-CoV-2 virus based on molecular profiles. The swab sample analysis system may be implemented as the swab sample analysis system as shown in FIGS. 1-2. In some examples, the swab sample analysis system may include a sampling device. In some instances, the sampling device may be configured as the sampling device 300 as shown in FIG. 3. The tip portion of the sampling device has an internal reservoir with a dimeter of 2.7 mm. In some instances, the diagnosis process 400 as shown in FIG. 4 is used.

In some instances, lipids with m/z values in a range of 600 and 1000 are the major molecular components of the viral envelope structure and are involved in key steps in their replication cycle. Although viruses bud from and acquire all lipids from different parts of their host membranes, viral lipid composition is specific to virus strains, and quantitatively distinct from the host membranes lipid composition. Viral lipid composition varies primarily based on budding site; the SARS-CoV-2 virus, for example, bud from the membrane of the host intermediate pre-Golgi compartment. In a study by Van Genderen et al (Biochem. Soc. Trans., 1995, 23 (3): 523-526), the viral envelope of the coronavirus murine hepatitis virus (MHV) shows a greater proportion of sphingomyelins, PS, and PI than the host cells, and a reduced proportion of PE. The proportion of PI in the viral membranes of MHV is increased by 4% from host cells and the proportion of PS/PI species is reduced by 12%. In some instances, lipid variation is also induced in the infected host cells, as viral pathogens remodel host lipid metabolism to enable replication. In a study by Yan et al (Viruses. 2019; 11(1). pii: E73), fatty acids and glycerophospholipids (e.g., lysoPC and lysoPE) are significantly increased in the human SARS-CoV-2 virus infected cells compared to healthy cells. In some implementations, dysregulation of highly abundant glycerophospholipids in infected host cells and the unique lipid composition of the pathogen itself may represent a promising detection target for diagnostic tests. In some instances, the methods and systems described here can provide high sensitivity and selectivity for untargeted chemical analysis of endogenous compounds such as metabolites, lipids, and proteins from pathogen/host with minimal sample preparation requirements. Using a tandem MS, various lipid markers as well as virus-specific proteins (e.g., SARS-CoV-2 nucleocapsid, N-protein) can be detected and identified. In some instances, host biomarkers can be detected which can potentially provide information on treatment option and prognosis.

FIG. 6 is a schematic diagram showing aspects of an example sampling device 600. As shown in FIG. 6, the example sampling device 600 includes a tip portion 602 and a housing 604. The tip portion 602 includes a mandrel end 606 having a tapered cylindrical shape, and a cylindrical end 608 engaged with a receiving end of the housing 604. In some implementations, the cylindrical end 608 seals with the receiving end of the housing 604 (e.g., forming an air-tight seal). In some examples, the example sampling device 600 may include additional or different components, and the components may be arranged as shown or in another manner.

As shown in a cross-sectional view of the tip portion 602 in FIG. 6, the tip portion 602 includes three distinct internal channels, including a liquid supply channel 612, a liquid extraction channel 614, and a gas channel 616. In some implementations, the three internal channels 612, 614, 616 may be implemented as the three internal channels 312, 314 and 316 as shown in FIG. 3 or in another manner. In some implementations, the housing 604 is implemented as the housing 304 as shown in FIG. 3 or in another manner.

In some cases, the liquid supply channel 612 may be implemented as the liquid supply channel 312 as shown in FIG. 3. In some implementations, the internal reservoir 618 may have a cylindrical shape and be coupled to the liquid supply channels 612. In certain examples, the liquid solvent received from the liquid supply channel 612 in the internal reservoir 618 makes direct contact with the swab head which may be inserted via an opening 626 at one end of the tip portion 602. In some instances, at least a portion of a specimen collected from a patient may be suspended such that molecules from cells in a virus may be extracted directly from the swab in the internal reservoir 618 into the liquid solvent. In some implementations, the diameter 622 of the internal reservoir 618 is determined by, for example, the size of the swab head and the possible amount of disease marker on the swab head. In some instances, the diameter 622 and height 624 of the internal reservoir 618 may determine the volume of the liquid solvent exposed to the swab head and performance aspects of the chemical measurement system, for example limit of detection, and accuracy. In some instances, the diameter 622 and height 624 of the internal reservoir 618 is designed for maximal extraction of a target material. In some instances, the diameter of the internal reservoir 618 of the tip portion 602 may be in a range of 1-20 mm. In certain instances, the internal reservoir 618 may have a different shape, aspect ratio, size, or dimension. In some instances, the liquid extraction channel 614 provides a second, distinct internal pathway 634 in the tip portion 602. In some cases, the liquid extraction channel 614 may be implemented as the liquid extraction channel 314 as shown in FIG. 3. In some implementations, the gas channel 616 provides a third, distinct internal pathway 636 in the tip portion 602. In some instances, the gas channel 616 may be implemented as the gas channel 316 as shown in FIG. 3 or in another manner.

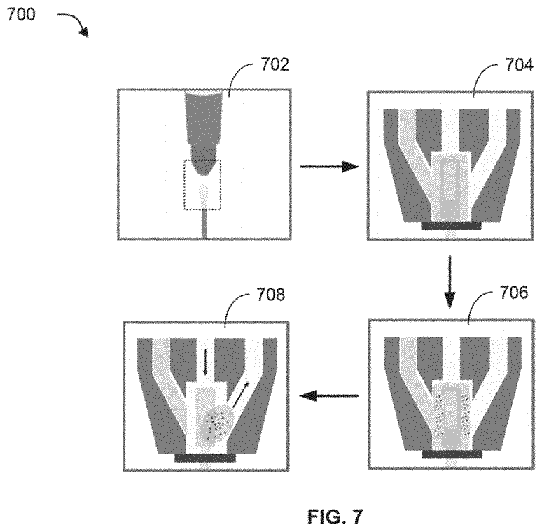

FIG. 7 is a flow chart showing an example swab sample analysis process 700. In some implementations, the example process 700 may be an automated process used for detection and identification of the SARS-CoV-2 virus or another disease marker. The example process 700 can be performed, for example, by a swab sample analysis system. For instance, operations in the example process 700 may be performed by the swab sample analysis systems shown in FIGS. 1-2 or another type of swab sample analysis system with additional or different components. In some instances, a sampling device of the swab sample analysis system may be implemented as the sampling device 600 or in another manner. The example process 700 may include additional or different operations, including operations performed by additional or different components, and the operations may be performed in the order shown or in another order. In some cases, operations in the example process 700 can be combined, iterated or otherwise repeated or performed in another manner.

At 702, a swab sample (e.g., a tip or portion of the swab containing the swab sample) is inserted into a tip portion of a sampling device. In some instances, the inserted swab sample contains a specimen collected from a patient. In some instances, the specimen may be collected using a nasal swab, nasopharyngeal swab, oropharyngeal swab, throat swab, or another type of swab. In some instances, swab samples may be categorized into four groups associated with four clinical courses, e.g., asymptomatic, mild, moderate, and severe. In some instances, other type of specimens with higher viral load may be collected and tested, e.g., sputum. In some instances, the swab head may be inserted into an internal reservoir of the tip portion. In some instances, after inserting the swab head into the tip portion through the opening, the tip portion may be sealed by a sealing element associated with the opening such as an O-ring, a membrane, or another type of sealing element such that when the tip portion is inserted into the internal reservoir, the internal reservoir can hold the liquid solvent in direct contact with the swab head without leaking.

At 704, a liquid solvent is supplied. In some implementations, the liquid solvent may be supplied to the swab head of the swab sample in the internal reservoir of the sampling device to mix material from the swab sample with the liquid solvent, e.g., to suspend viruses and/or host cells, and to extract molecules from the swab head. In some implementations, the liquid solvent is supplied to the internal reservoir with controlled volume and flow rate. In some implementations, a control system (e.g., the control system 204 as shown in FIG. 2) may be used to supply the liquid solvent. In some implementations, the control system may be controlled by a control unit (e.g., the control unit 224 as shown in FIG. 2).

At 706, an analyte is formed. In some implementations, the liquid solvent after being delivered to the internal reservoir of the sampling device may interact with at least a portion of the specimen in the swab sample and form the analyte in the internal reservoir. In some instances, the analyte may include one or more of the following: pathogen molecules, molecules from cells of the host and molecules in secretions produced by the cells of the host. In some implementations, the liquid solvent may be allowed to interact with the swab head for a certain time period. In some implementations, the time period may be determined by one or more of a type of solvent (e.g., polar or non-polar) and temperature. In some instances, the analyte may be formed in the internal reservoir after the time period.

At 708, the analyte is extracted. In some implementations, the extraction of the analyte from the internal reservoir may be performed by applying a pressure on one end of a transfer tube coupled to the sampling device. In some instances, the pressure may be lower than the atmospheric pressure. In some implementations, the analyte may be extracted into and analyzed by a mass spectrometer of the swab sample analysis system. In certain examples, prior to being analyzed by the mass spectrometer, the analyte may be ionized using an electrospray ionization or in another manner. In some instances, the mass spectrometer of the swab sample analysis system includes an Orbitrap QE Mass Spectrometer operating under negative ion mode with a resolving power: 140,000 and a mass accuracy <5 ppm. In some instances, after extraction, all the channels and the internal reservoir in the probe device and the transfer tubes attached to the sampling device may be thoroughly flushed one or more times with a cleaning solvent following a washing procedure, or discarded to avoid potential contaminations.

In some implementations, the mass spectrometry data obtained by the mass spectrometer may be imported into a commercial statistical analysis software, e.g., RStudio. In some instances, after importing, the mass spectral data may be binned to m/z=0.01, and normalized to a total ion current. In some instances, background including molecular features originating from the liquid solvent may be subtracted from the mass spectrometry data. In some implementations, a principle component analysis is performed on the processed mass spectrometry data and results including classification can be returned. For example, a prcomp function in RStudio may be used. In some instances, multiple samples (e.g., n=200) including samples with other pathogens and microorganisms may be collected and used to evaluate specificity. In some instances, intra- and inter-sample experiments with control samples/medical swabs may be performed, e.g., n=20/prototype.

In some instances, molecular features in mass spectra obtained from samples collected from coronavirus-positive patients may be associated with their clinical outcome, including asymptomatic, mild (recovery at home), moderate (non-ICU hospitalization), and severe (ICU hospitalization) clinical course. In some instances, the statistical classification model may be employed to build the four-class statistical classifier using the mass spectrometry data obtained from the swab samples. In some instances, performance of the statistical classification model is assessed using cross-validation by evaluating re-call for each class (e.g., an accuracy of greater than 85%). Predictive markers may be identified using tandem MS and high mass accuracy measurements. In some instances, host molecular markers including lipids and metabolites that are indicative of clinical course for the coronavirus-positive patients may be identified. In some instances, robust predictive models can be constructed to accurately diagnose the coronavirus disease and other related pathogens, and to identify novel predictive markers of the disease and of host response.

In some instances, the system and method may have a reproducibility with a relative standard deviation (RSD) of less than 5%, a sensitivity with a total ion count greater than 1×10⁵ and a signal-to-noise ratio (S/N) greater than 10 for molecular analysis, and a total analysis time of less than 30 seconds. In some instances, the system and method may have a sensitivity of greater than 95%, a false-positive rate of less than 5%, a selectivity of greater than 80%, a false-negative rate of less than 20%, and an accuracy of greater than 90%.

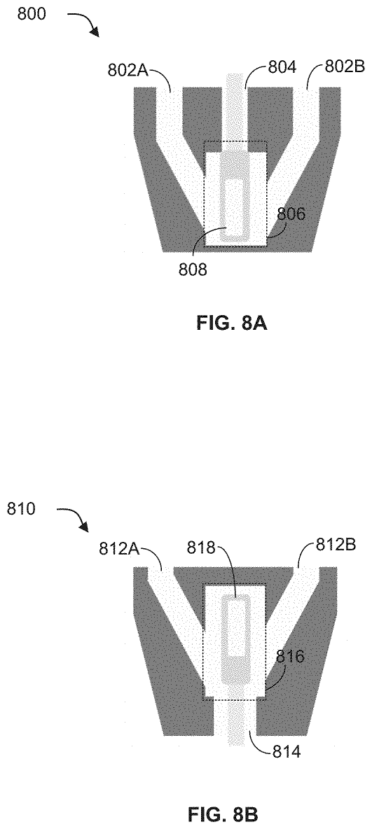

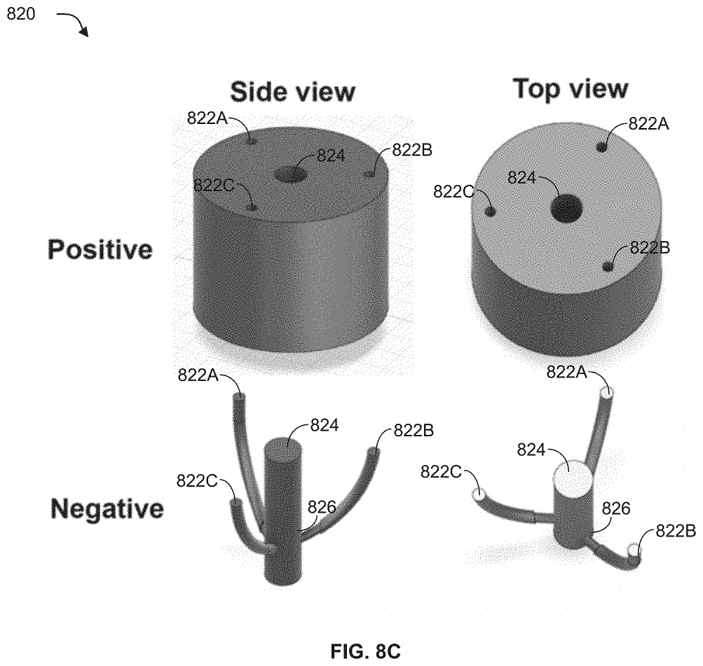

FIGS. 8A-8C are schematic diagrams showing cross-sectional views of example tip portions that may be used in a swab sample analysis system. In some instances, the tip portions 800, 810, 820 may be integrated with a sampling device, e.g., the sampling devices 600 as shown in FIG. 6. As shown in FIG. 8A, the tip portion 800 includes two distinct internal channels including a liquid supply channel 802A and a liquid extraction channel 802B. In some instances, the liquid supply/extraction channels 802A, 802B may be implemented as the liquid supply/extraction channels 612, 614 as shown in FIG. 6 or in another manner. The tip portion 800 further includes an internal reservoir 806, which may be implemented as the internal reservoir 618 as shown in FIG. 6. The tip portion 800 further includes a swab port 804, on the same end as the liquid supply/extraction channels 802A, 802B terminate. In some instances, a swab head 808 may be inserted through the swab port 804 into the internal reservoir 806 for testing.

As shown in FIG. 8B, the tip portion 810 includes two distinct internal channels including a liquid supply channel 812A and a liquid extraction channel 812B. In some instances, the liquid supply/extraction channel 812A, 812B may be implemented as the liquid supply/extraction channel 612, 614 as shown in FIG. 6 or in another manner. The tip portion 810 further includes an internal reservoir 816, which may be implemented as the internal reservoir 618 as shown in FIG. 6. The tip portion 810 further includes a swab port 814 which is on the opposite end to the end where the liquid supply/extraction channels 812A, 812B terminate. In some instances, a swab head 818 may be inserted through the swab port 814 into the internal reservoir 816 for testing.

As shown in FIG. 8C, the tip portion 820 includes three distinct internal channels including a liquid supply channel 822A, a liquid extraction channel 822B and a gas channel 822C. In some instances, the liquid supply/extraction channel 822A, 822B, 822C may be implemented as the liquid supply/extraction channel/gas channel 612, 614, 616 as shown in FIG. 6 or in another manner. The tip portion 820 further includes an internal reservoir 826, which may be implemented as the internal reservoir 618 as shown in FIG. 6. The tip portion 820 further includes a swab port 824 which is on the same end as the end where the liquid supply/extraction/gas channels 822A, 822B, 822C terminate. In some instances, a swab head (not shown) may be inserted through the swab port 824 into the internal reservoir 826 for testing. In some instances, the liquid supply/extract channels 822A, 822B, the gas channel 822C and the swab port 824 may be arranged in another manner.

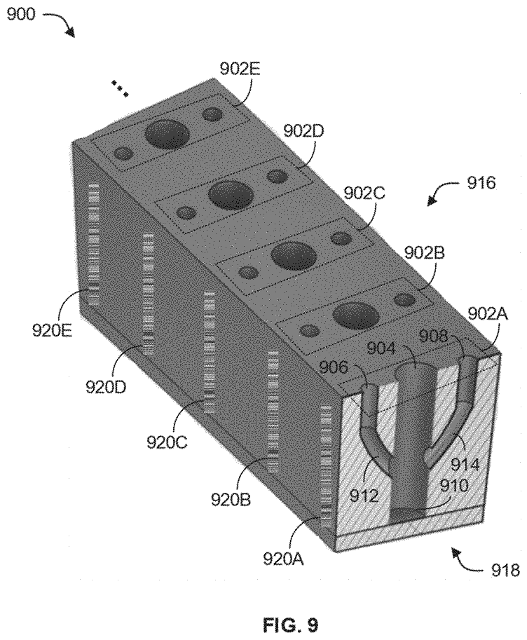

FIG. 9 is a schematic diagram showing aspects of an example sampling cartridge 900 that can be used, for example, in a swab sample analysis system. In some instances, the sampling cartridge 900 can be used for high-throughput testing and screening. The example sampling cartridge 900 shown in FIG. 9 includes multiple test units 902 for receiving multiple swab samples. As shown in FIG. 9, the sampling cartridge 900 includes 5 test units (e.g., 902A, 902B, 902C, 902D, and 902E) for holding and testing 5 swab samples. A sampling cartridge may include another number of test units (e.g., 5, 10, 15, 20, 50, 100, etc.). In the example shown, each of the test units includes a swab port 904, a liquid supply port 906, a liquid extraction port 908, an internal reservoir 910, a liquid supply channel 912, and a liquid extraction channel 914. As shown in FIG. 9, each of the swab port 904, the liquid supply port 906 and the liquid extraction port 908 can be accessed from a first surface 916 of the sampling cartridge 900. In some instances, the swab port 904 may be accessed from the first surface 916; and the liquid supply/extraction ports 906, 908 may be accessed from a second, opposing surface 918 of the sampling cartridge 900 or in another manner. In some instances, each of the test units 902 may be labeled to correlate the swab ports with the swab samples from patients. As shown in FIG. 9, each of the five test units 902 may be labeled with a respective barcode 920.

In some instances, the liquid supply port 906 and the liquid extraction port 908 are configured to provide fluidic communication with a control system and a mass spectrometer through respective transfer tubes. In some implementations, the sampling cartridge 900 may be composed of biologically compatible synthetic polymers. In some implementations, the sampling cartridge 900 may be fabricated using a 3D printing process, a machining process, or another type of fabrication process. In some instances, the swab port 904 in a test unit 902 can receive a swab head sample which can be held in place in the internal reservoir 910.

In some aspects of operation, the liquid supply channel 912 in a test unit 902 receives the liquid solvent from an external container, guides the liquid solvent to the internal reservoir 910, where the liquid solvent may be in direct contact with the swab head sample, and fills at least a portion of the internal reservoir 910 with the liquid solvent. In some implementations, the liquid solvent may be received from the external container as a part of a control system, e.g., the control system 204 as shown in FIG. 2 or another type of control system. In some instances, the control system may include a multi-channels mechanical pumping system providing individually addressable control of each fluidic channel for each of the test units 902. In some instances, the control system may also include multiple valves which are attached to respective transfer tubes to control liquid motion into or out of each of the test units 902 to automatically and sequentially analyze the swab samples in the sampling cartridge.

In some implementations, the internal reservoir 910 in a test unit 902 may have a cylindrical shape and be coupled to the liquid supply channels 912. In certain examples, the liquid solvent received from the liquid supply channel 912 in the internal reservoir 910 makes direct contact with the swab head sample inserted from the swab port 904 and at least a portion of the specimen on the swab head sample is extracted into the liquid solvent. In some instances, dimensions of the internal reservoir 904 is designed to receive the entire swab head for maximal molecular extraction.

In some instances, the liquid extraction channel 914 in a test unit 902 obtains an analyte by extracting at least a portion of liquid solvent carrying extracted molecules from the internal reservoir 910, and guides the analyte to the transfer tube that is coupled to a mass spectrometer. In some implementations, the analyte from the internal reservoir 910 may be extracted by a vacuum pump coupled to the mass spectrometer. In some implementations, a low pressure created on one end of the transfer tube may facilitate liquid aspiration to drive the analyte from the internal reservoir 910 to the mass spectrometer through the liquid extraction channel 914.

During operation, one or more swab samples can be simultaneously loaded to the test units 902 on the sampling cartridge 900 and inserted into the internal reservoirs 910 of the test units 902. In some instances, the swab samples are analyzed following a pre-programmed sequence. For example, a first internal reservoir 910A of a first test unit 902A may be filled with a liquid solvent (e.g., a mixture of chloroform and ethanol); the first internal reservoir 910A stays filled for 3-10 seconds or another time period for suspension of viruses/host cells and extraction of molecules from the viruses/host cells from the swab sample; and a first analyte from the first test unit 902A may be transferred to a mass spectrometer for analysis. In some implementations, the time period may be determined by one or more of a type of solvent (e.g., polar or non-polar) and temperature. In some examples, the cartridge 900 can be used to reduce lag time between switching samples, to perform uninterrupted sample analysis, and to improve the screening throughput of the swab sample analysis system. For example, when the first analyte is being transferred to the mass spectrometer, the liquid solvent can be delivered simultaneously to a second internal reservoir 910B of a second test unit 902B. In some instances, assuming testing of a swab sample takes about 30 seconds, testing and screening 10 swab samples using a sampling cartridge 900 with 10 test units 902 can be completed in 5 minutes.

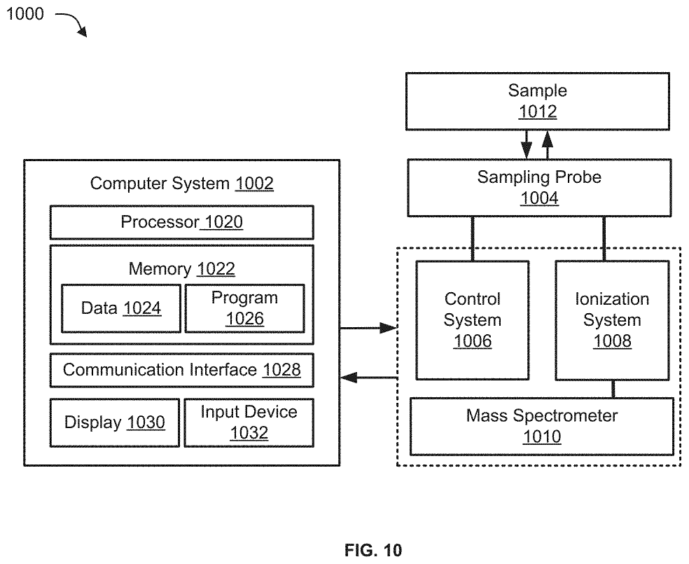

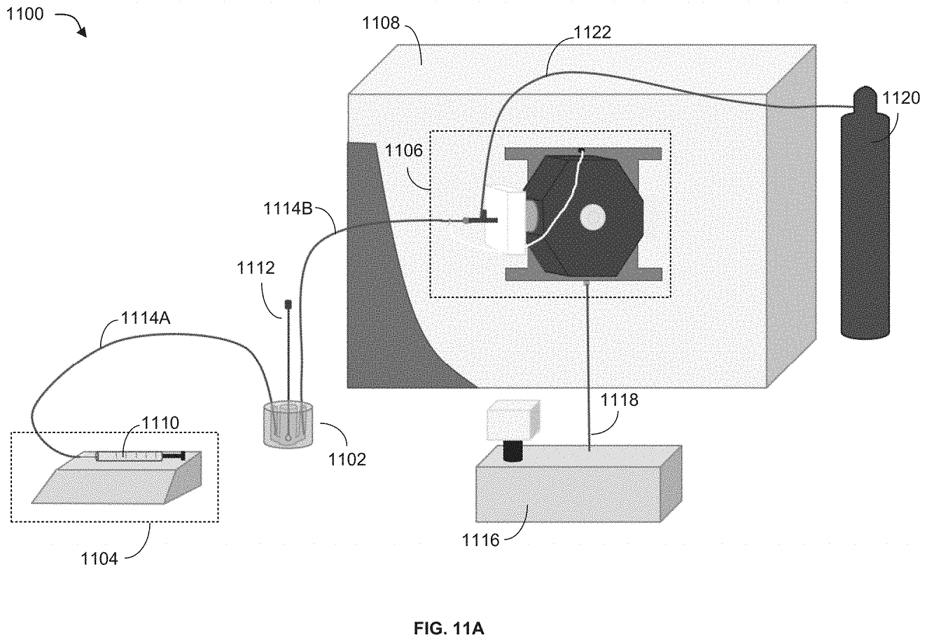

FIG. 10 is a schematic diagram of an example swab sample analysis system 1000. As shown in FIG. 10, the example system 1000 includes a computer system 1002, a sampling device 1004, a control system 1006, an ionization system 1008, and a mass spectrometer 1010. In some implementations, the example system 1000 may be used for qualitatively and quantitatively identification and classification of specific markers of disease (e.g., the SARS-CoV-2 virus and its various strains, or other types of diseases). In some implementations, the example system 1000 is used in a laboratory environment to evaluate biological samples collected using swabs, or in another manner. In some instances, the example system 1000 can be used for on-site testing and screening. In some examples, the example system 1000 may include additional or different components, and the components may be arranged as shown or in another manner.

In the example shown in FIG. 10, the computer system 1002 includes a processor 1020, memory 1022, a communication interface 1028, a display device 1030, and an input device 1032. In some implementations, the computer system 1002 may be implemented and operated as the computer system 102 shown in FIG. 1 or in another manner. In some implementations, the sampling device 1004 may be configured to communicate fluids with the control system 1006, a sample 1012 (e.g., a swab sample), and the ionization system 1008 via respective transfer tubes. In some implementations, the sampling device 1004 may be implemented as the example sampling devices 300, 600, 800, 810, and 820 shown in FIGS. 3, 6, and 8A-8C. In certain implementations, the sampling device 1004 may be implemented as the example cartridge 900 shown in FIG. 9 or in another manner. In some implementations, the mass spectrometer 1010 may be implemented as the mass spectrometer 108 shown in the example system 100 of FIG. 1 or in another manner.

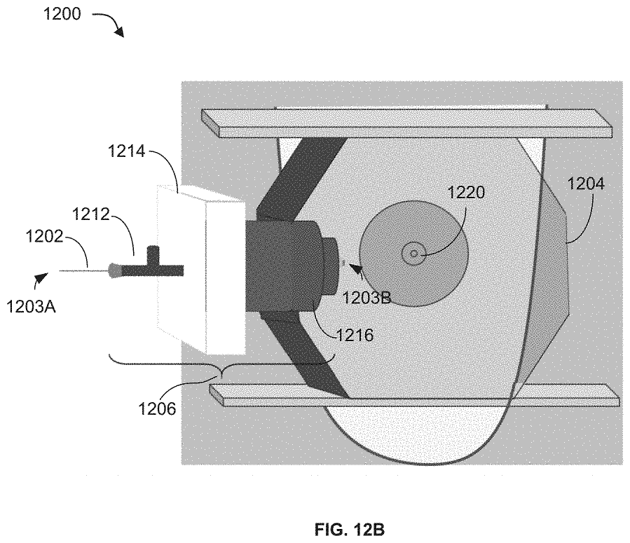

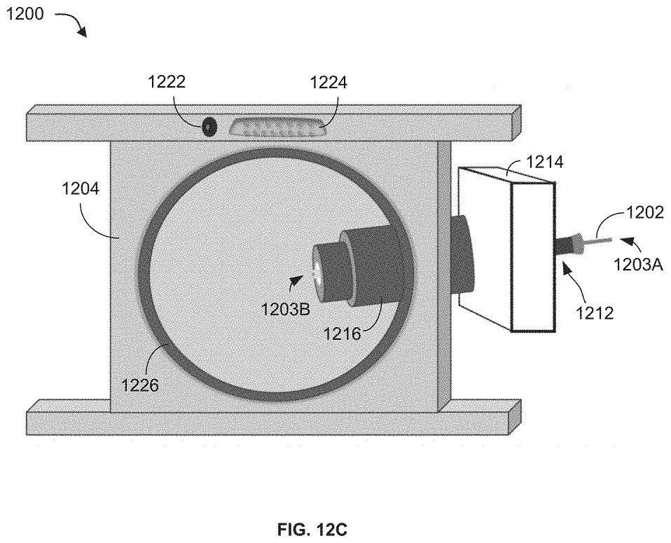

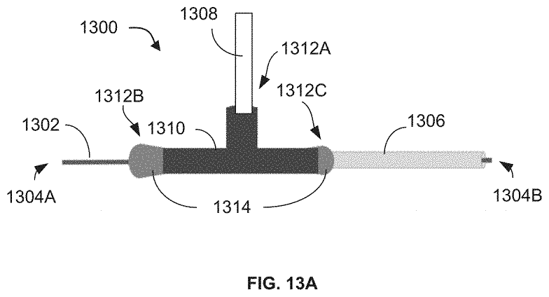

In some implementations, the ionization system 1008 may include an electrospray ionization source or another ionization source. In some examples, the ionization system 1008 may include a transfer capillary with one end enclosed in an chamber and with the other end directly coupled to the sampling device 1004 via a transfer tube. In some instances, a valve may be coupled on the transfer tube or the transfer capillary to control the flow of an analyte. In some implementations, the valve may be controlled by the control system 1006. In some examples, the chamber can be coupled to a vacuum pump, which can be used to create a low pressure in the chamber. In certain examples, the chamber may be integrated to the mass spectrometer and the low pressure in the chamber may be created by a vacuum system of the mass spectrometer or in another manner. In some implementations, the low pressure may facilitate the extraction of the analyte from the sampling device 1004 to the chamber. A discharge voltage (e.g., up to a few kilovolt) can be applied on the transfer capillary to obtain an ionized analyte. The ionized analyte containing a gas cluster of ionization products of the liquid solvent and dissolved chemical compounds from the sample 1012 can be delivered to the mass spectrometer 1010 for analysis. In some examples, the ionization system 1008 may be implemented as the example ionization system 1200 as shown in FIGS. 12A-12C or in different manner. In certain examples, the transfer capillary and the chamber may be assembled together as shown in FIGS. 12A-12C, or in another manner, such as a commercial electrospray ionization source/sprayer. In some instances, the ionization system 1008 can be used to improve the ionization efficiency, sensitivity, and reproducibility of the example system 1000.

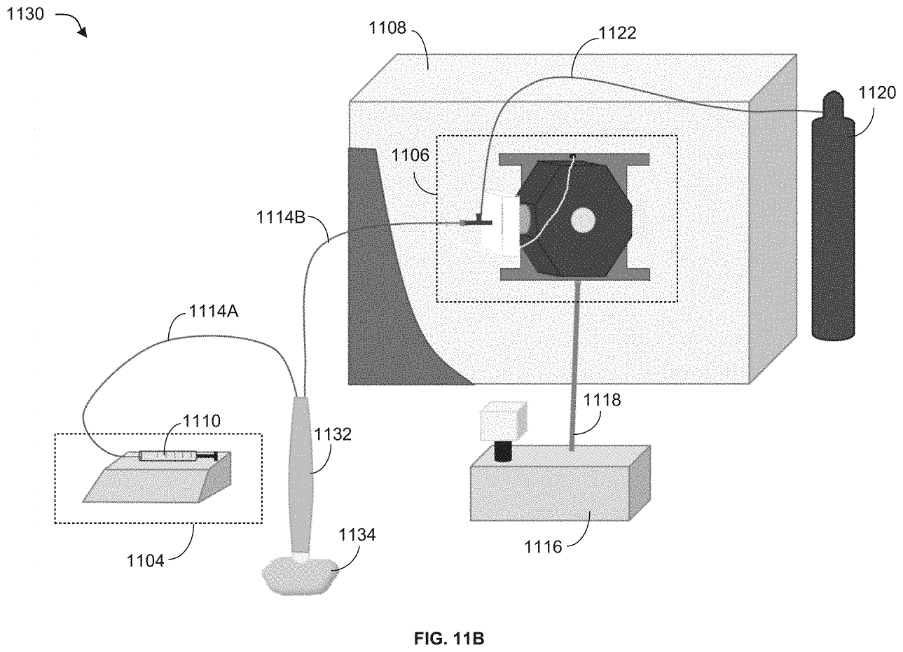

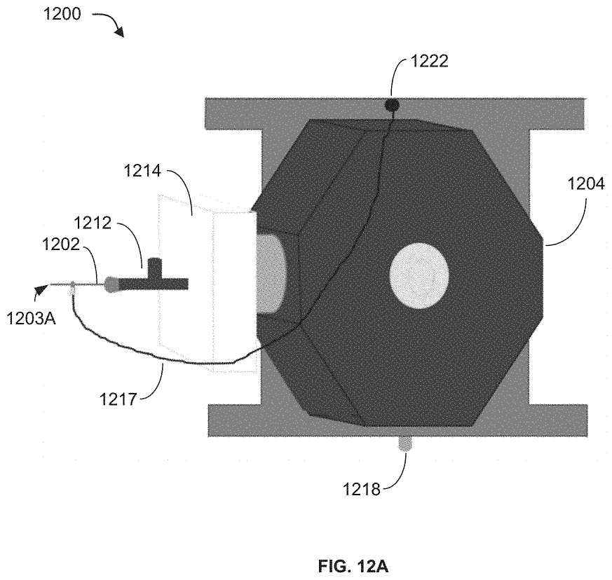

FIGS. 11A-11B are schematic diagrams showing aspects of example swab sample analysis systems 1100 and 1130. As shown in FIGS. 11A-11B, the example systems 1100 and 1130 include a mechanical pumping system 1104, an ionization system 1106 and a mass spectrometer 1108. The example system 1100 includes a sampling device 1102 for receiving and analyzing a swab sample 1112. The example system 1130 includes a sampling device 1132 for analyzing a specimen extracted from a swab sample and deposited on a substrate surface 1134. In some implementations, the sample surface 1134 may be another type of surfaces, for example in-vivo or ex-vivo tissue samples that are directly analyzed with sampling device 1132. As shown in FIGS. 11A and 11B, each of the sampling devices 1102 and 1132 is coupled between an external container 1110 in the mechanical pumping system 1104 and the ionization system 1106 through respective transfer tubes 1114A, 1114B. In some instances, the example systems 1100 and 1130 may be adapted to enable sensitive and robust analysis of swab samples. In some instances, the mechanical pumping system 1104, the mass spectrometer 1108, and the transfer tubes 1114A, 1114B may be implemented as the respective components in the example system 200 of FIG. 2 or in another manner. In some instances, the sampling device 1102 may be implemented as the example sampling devices 600, 800, 810, and 820 shown in FIGS. 6, and 8A-8C, the example sampling cartridge 900 shown in FIG. 9, or in another manner. In some instances, the sampling device 1132 may be implemented as the example sampling devices 300 shown in FIG. 3, may be operated according to the example process 400 for analyzing specimens extracted from swab samples and deposited on the surface 1134, or in another manner. In some examples, the example systems 1100 and 1130 may include additional or different components, and the components may be arranged as shown or in another manner.

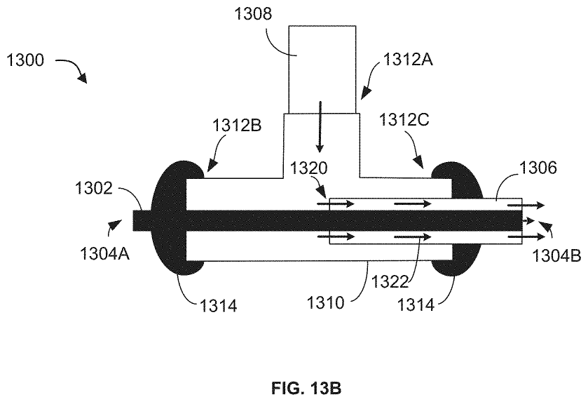

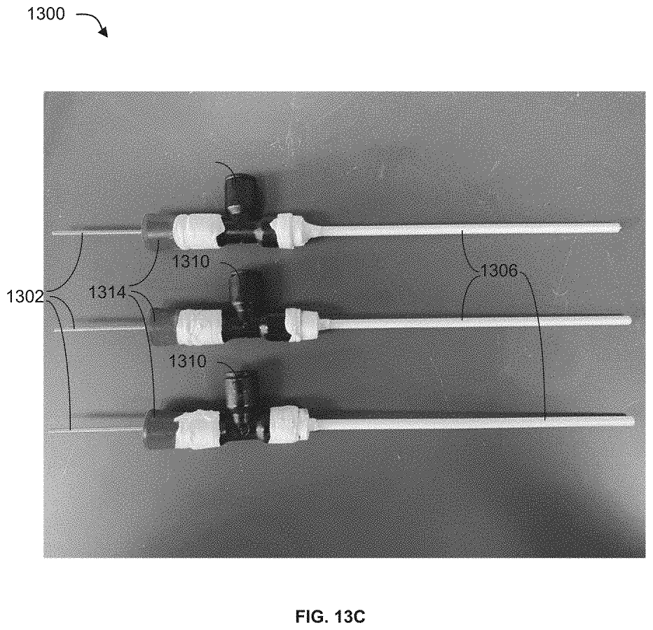

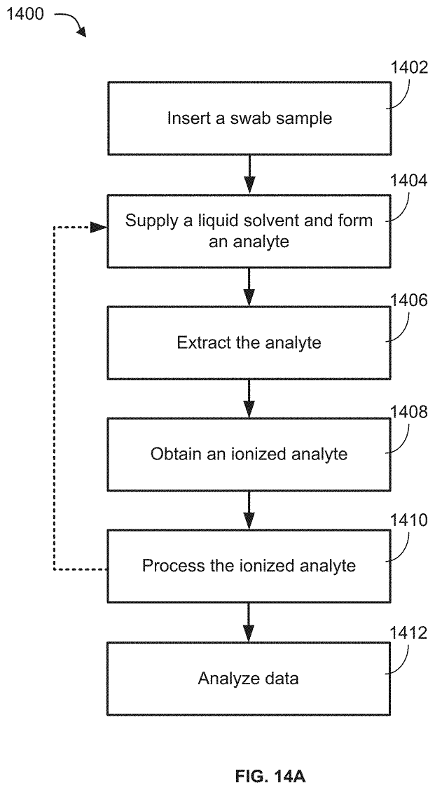

In some implementations, the ionization system 1106 may include an electrospray ionization (ESI) source or another ionization source. In some instances, the ionization system 1106, which may be implemented as the example ionization system 1200 shown in FIGS. 12A-12C, may include an ionization source and an chamber assembled together. In some implementations, the ionization source may be implemented as the ionization source 1300 shown in FIGS. 13A-13B or in another manner. The transfer capillary can be coupled with the sampling device 1102 or 1132 via the transfer tube 1114B and with the chamber, which enables a fluidic communication of an analyte from the sampling device 1102/1132 to the chamber. The chamber of the ionization system 1106 may be vacuum sealed and pumped to a low pressure by a vacuum pump 1106 via a vacuum line 1118 to facilitate the extraction of the analyte from the sampling device 1102/1132 to the chamber. In some instances, the low pressure in the chamber may be in a range of 1.0 to 2.0 mbar, or in another range according to the mass spectrometer, vacuum settings, and another parameter. As shown in FIGS. 11A and 11B, the ionization system 1106 is directly integrated with the mass spectrometer 1108 so that an ionized analyte produced in the chamber can be directly transferred to the mass spectrometer 1108 for analysis.

As shown in FIGS. 11A and 11B, the ionization system 1106 is coupled to a gas line 1122 to receive carrier gas from a gas container 1120. In some implementations, the ionization source may be configured to receive the carrier gas and to create a coaxial sheath gas around a portion of the transfer capillary. In some instances, the coaxial sheath gas can be used to guide the analyte extracted from the transfer capillary, for example, toward an inlet tube of the mass spectrometer 1108, and improve evaporation and nebulization. In some implementations, the transfer capillary can be heated by a heating element to facilitate the vaporization of the analyte. In some implementations, the transfer capillary is an electrically conductive tube (e.g., a 316 stainless-steel tube) and a discharge voltage (e.g., several kilovolts) to generate an electric field for ionization can be applied on the transfer capillary. The electric field may cause dispersion of the analyte into an ionized analyte in the chamber, which may be in a form of an aerosol of highly charged droplets with ionized species in gas phase. The ionized analyte can then be delivered to the mass spectrometer 1108 for analysis.

As shown in FIG. 11B, the example sample analysis system 1130 includes an external container 1110, an electrospray ionization system 1106, a mass spectrometer 1108, one or more computer systems, a sampling device 1132, and a control system. The external container 1110 includes a liquid solvent. The electrospray ionization system 1106 is configured to ionize an analyte. The mass spectrometer 1108 is configured to produce mass spectrometry data by processing the ionized analyte provided by the electrospray ionization system 1106. The sampling device 1132 includes an internal reservoir, an inlet channel, a gas channel, and an outlet channel. In some instances, the sampling device 1132 is implemented as the sampling device 202, 300 as shown in FIGS. 2-3. The internal reservoir is configured to hold a fixed volume of the liquid solvent in direct contact with a surface of a sample 1134 for a period of time to form the analyte in the sampling device. In some instances, the fixed volume is defined by the volume of the internal reservoir or in another manner. The sample is a biological tissue or another type of sample.

The electrospray ionization system 1106 includes a heating element attached to the transfer capillary. The heating element can heat at least a portion of the transfer capillary and thus the analyte in the transfer capillary. The analyte upon being heated can be vaporized. In some instances, the transfer capillary is electrically conductive. A discharge voltage is applied on the conductive transfer capillary and the analyte can be ionized. The ionized analyte can be aspirated into the chamber of the electrospray ionization system 1106 through a tip of the transfer capillary in the chamber. The mass spectrometer includes an inlet tube residing in proximity to the tip of the transfer capillary in the chamber. A low pressure can be applied on the inlet tube to extract the ionized analyte into the mass spectrometer. In some instances, a gas sheath can be formed around at least a portion of the transfer capillary, for example through a coupler (e.g., the example coupler 1300 shown in FIGS. 13A-13C) to guide the ionized analyte to the inlet tube of the mass spectrometer. The electrospray ionization system 1106 can be implemented as the example ionization system 1200 shown in FIGS. 12A-12C, or in another manner.

FIGS. 12A-12C are schematic diagrams showing aspects of an example ionization system 1200. The ionization system 1200 includes an chamber 1204 and an ionization source 1206 which includes a transfer capillary 1202, a coupler 1212, a support block 1214 and a heating element 1216. In some implementations, the example ionization system 1200 may be used to ionize both polar and non-polar molecules in liquid solvent or chemical compounds dissolved from a swab sample, where positively or negatively charged ions and/or analyte ions can be generated. In some instances, the example ionization system 1200 may be implemented as the ionization system 1106 in the example systems 1100 and 1130 as shown in FIGS. 11A and 11B. In some implementations, the example ionization system 1200 can be directly coupled to a mass spectrometer, for example, the mass spectrometer 1108 shown in FIGS. 11A-11B. In some examples, the example ionization system 1200 may include additional or different components, and the components may be arranged as shown or in another manner.

As shown in FIGS. 12A-12C, the ionization source 1206 is an electrospray ionization source. The transfer capillary 1202 has a first end 1203A and a second end 1203B. The first end 1203A is located outside the chamber 1204 and may be fluidically coupled to a sampling device through a transfer tube, e.g., the sampling device 1102/1132 through the transfer tube 1114B as shown in FIGS. 11A and 11B. The second end 1203B is enclosed in the chamber 1204.

In some examples, the support block 1214 in the ionization source includes an insulating material (e.g., ceramic) and can be used to provide mechanical support to the transfer capillary 1202 and the coupler 1212. In certain instances, the support block 1214 allows the transfer capillary 1202 and the coupler 1212 to be structurally integrated with the chamber 1204. For example, the support block 1214 can be retrofitted and fused to one end of the heating element 1216. As shown in FIGS. 11A-11C, a hole on the support block 1214 can be configured to guide and orient the transfer capillary 1202 so that the second end 1203B of the transfer capillary 1202 can be aligned with the inlet tube 1220 of the mass spectrometer. In certain examples, when the coupler 1212 is assembled on the support block 1214, the chamber 1204 can be vacuum sealed. In certain examples, the heating element 1216 can be used to heat at least a portion of the transfer capillary 1202 to increase the temperature of the analyte traveling in the transfer capillary 1202 and to assist in the vaporization of the analyte at the second end 1203B. In some implementations, the ionization source 1206 may include additional or different components, and the components may be arranged as shown or in another manner.

In some implementations, the chamber 1204 is integrated to the mass spectrometer and aligned with the inlet tube 1220 and forms a vacuum seal against the housing of the mass spectrometer using an O-ring 1226. In some implementations, a vacuum pump may be coupled at a vacuum port 1218 on the chamber 1204. The vacuum pump can be used to create a low pressure in the chamber 1204. In some implementations, aspiration of an analyte from the sampling device to the chamber 1204 across the transfer capillary 1202 may be facilitated by the low pressure created in the chamber 1204.

In some implementations, the transfer capillary 1202 includes an electrically conductive tube, for example, a 316 stainless-steel tube. In some implementations, the chamber 1204 may include metal, e.g., aluminum (Al) As shown in FIGS. 12A-12C, the transfer capillary 1202 can be electrically coupled to an electrical port 1222 through a conductive connection 1217 to receive a DC voltage. The DC voltage (e.g., up to 8 kV or another voltage) is applied on the transfer capillary 1202 for ionizing the analyte extracted from the transfer capillary 1202 into the chamber 1204. In some examples, a DC voltage of 4-6 kV is applied during the time of the extraction of the analyte. When the DC voltage is applied on the transfer capillary 1202, the analyte containing the liquid solvent and dissolved chemical compounds are excited and ionized creating an ionized analyte. In some examples, the ionized analyte including analyte ions may be formed in the chamber 1204. In some implementations, the ionization source 1206 and the mass spectrometer may be communicably coupled via a control port 1224 allowing control and communication between the ionization source and the mass spectrometer.

In some implementation, the second end 1203B of the transfer capillary 1202 may be configure in proximity to the inlet tube 1220. For example, the second end 1203B of the transfer capillary 1202 and the inlet tube 1220 of the mass spectrometer are closely arranged and separated by a distance (e.g., a few millimeters to centimeters) so that the ionized analyte after forming can be transfer out to the mass spectrometer through the inlet tube 1220. In some implementations, the ions can be captured and analyzed by the mass analyzer within the mass spectrometer, e.g. an orbitrap mass analyzer or an ion trap mass analyzer. In some implementations, prior to the mass analyzer, the ionized analyte may be collected and transferred through ion optic systems (e.g., ion guides such as an 5-lens, or a quadrupole/hexapole). In some instances, the ion optic system may be configured to filter neutral species in the ionized analyte and allow ions passing through. In some instances, the ion optic system may be used to eliminate contamination of the mass spectrometer.

FIGS. 13A-13B are schematic diagrams showing aspects of an example ionization source 1300 in an ionization system. The example ionization source 1300 includes a transfer capillary 1302, a ceramic tube 1306 and a coupler 1310. The example ionization source 1300 may be integrated to an chamber, e.g., the chamber 1204 of the example ionization system 1200. The transfer capillary 1302 includes a first end 1304A and a second end 1304B. In some implementations, the first end 1304A of the transfer capillary 1302 may be implemented as the first end 1203A of the transfer capillary 1202, which can be fluidically coupled to a transfer tube for receiving an analyte from a sampling device. In some implementations, the second end 1304B of the transfer capillary 1302 may be implemented as the second end 1203B of the transfer capillary 1202, which may be enclosed in the chamber 1204. As shown in FIGS. 13A and 13B, the transfer capillary 1302 is coupled to the ceramic tube 1306 and a gas tube 1308 via the coupler 1310. In some implementations, the ionization source 1300 may be detachable from the chamber, which can be disposed, cleaned, or replaced if contaminated, e.g., after a certain number (e.g., one or more) of regular uses or when switching between different samples. FIG. 13C shows a photograph of three example ionization sources 1300.

As shown in the example ionization source 1300, the coupler 1310, which is a T-shaped gas fitting, has three ports, e.g., a first port 1312A, a second port 1312B and a third port 1312C. In some example, the coupler 1310 is made of plastic, insulating polymers, rubber, ceramic, or another insulating material. The gas tube 1308 is fluidically coupled between the first port 1312A and a gas container to deliver carrier gas to the first port 1312A. In some instances, the carrier gas received from the first port 1312A may include inert or non-reactive gas such as nitrogen, argon, helium. The transfer capillary 1302 is organized across the second and third ports 1312B and 1312C. As shown in FIGS. 13A-13B, a portion of the transfer capillary 1302 between the first and second end 1304A, 1304B is coaxially enclosed in the ceramic tube 1306. As shown in the example ionization source 1300, the ceramic tube 1306 extends from inside of the coupler 1310 to the second end 1304B of the transfer capillary 1302. As shown in FIGS. 13A-13B, the second port 1312B is sealed against outer walls of the transfer capillary 1302, and the third port 1312C is sealed against outer walls of the ceramic tube 1306 by respective sealing elements 1314, e.g., rubber stoppers.

During operation, the carrier gas, after entering the first port 1312A of the coupler 1310, flows through the body of the coupler 1310 and enters an opening 1320 located inside of the coupler 1310 defined by the ceramic tube 1306 and the transfer capillary 1302. The carrier gas then flows through the spacing between the outer wall of the transfer capillary 1302 and the inner wall of the ceramic tube 1306 and exits on the opposite end of the ceramic tube 1306 located in approximation to the second end 1304B of the transfer capillary 1302. A gas stream 1322 inside the ceramic tube 1306 forms a coaxial gas sheath around the transfer capillary 1302 and flows into the chamber. In some examples, an analyte may be extracted from a sampling device in contact with a swab sample to the first end 1304A, across the transfer capillary 1302, and further to the second 1304B. The analyte aspirated out of the second end 1304B into the chamber can be ionized generating an ionized analyte. In some instances, the coaxial sheath gas exiting the end of the ceramic tube 1306 may be used to evaporate droplets of the extracted and ionized analyte at the second end 1304B and guide the ionized analyte from the second end 1304B of the transfer capillary 1302, for example toward the inlet tube 1220 of the mass spectrometer.

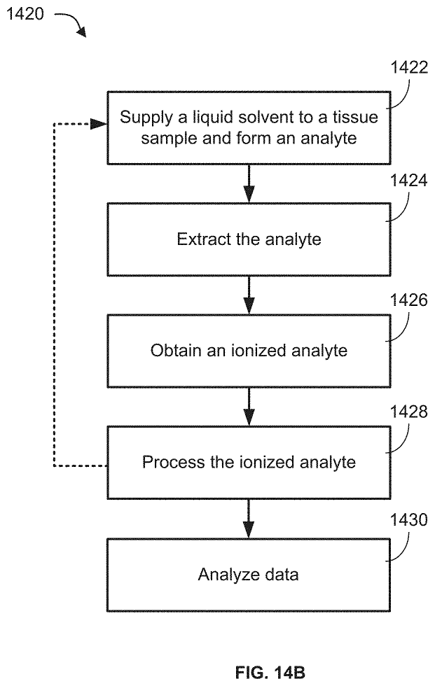

FIG. 14A is a flow chart showing an example swab sample analysis process 1400. In some implementations, the example process 1400 may be an automated process used for detection and identification of the SARS-CoV-2 virus or another disease marker. The example process 1400 may be performed, for example, by a swab sample analysis system. For instance, operations in the example process 1400 may be performed by the swab sample analysis systems shown in FIGS. 10, and 11A, or another type of swab sample analysis system with additional or different components. The example process 1400 may include additional or different operations, including operations performed by additional or different components, and the operations may be performed in the order shown or in another order. In some cases, operations in the example process 1400 can be combined, iterated or otherwise repeated or performed in another manner.

At 1402, a swab sample (e.g., a tip or portion of the swab containing the swab sample) is inserted into a sampling device. In some implementations, the operation 1402 may be implemented with respect to the operation 702 shown in the example process 700. At 1404, a liquid solvent is supplied, and an analyte is formed. In some implementations, a control system (e.g., the control system 1006 as shown in FIG. 10) may be used to supply the liquid solvent from an external container to an internal reservoir of the sampling device. In some examples, the liquid solvent may stay in the internal reservoir of the sampling device for a time period (e.g., 3-15 seconds) depending on amount of samples on the swab, solubility of the samples in the liquid solvent, type of liquid solvent, type and material of the swab, temperature and another parameter. In some implementations, the operation 1404 may be implemented with respect to the operations 704 and 706 shown in the example process 700.

At 1406, the analyte is extracted. In some implementations, the analyte may be extracted and transported by operation of an ionization system. In some implementations, the transportation of the analyte from the internal reservoir of the sampling device may be performed by applying a pressure on one end of a transfer tube coupled to the sampling device. In some instances, the pressure lower than the atmospheric pressure may be provided by an chamber of the ionization system. For example, the chamber may be under vacuum. In some examples, the analyte may be transported to an electrospray ionization source and sprayed from one end of a transfer capillary to the chamber. The ionization system may be implemented as the ionization system 1200 in FIGS. 12A-12C with an ionization source, e.g., the ionization source 1300 shown in FIGS. 13A-13B, or in another manner.

At 1408, an ionized analyte is obtained. In some implementations, the analyte extracted and sprayed into the ionization system may be ionized. In some examples, the ionization process may be operated under a pressure a range of 0.1-2 mbar, or another pressure. In some implementations, the ionized analyte may be obtained in the chamber by applying a discharge voltage on the transfer capillary. In some implementations, the ionized analyte may contain a gas cluster of ionization products of the liquid solvent and the dissolved chemical compound extracted from the swab sample. The ionized analyte may be evaporated and guided to an inlet tube of the mass spectrometer by a coaxial sheath gas created by the ionization source.

At 1410, the ionized analyte is processed. In some implementations, the ionized analyte may be collected and analyzed by a mass spectrometer, e.g., the mass spectrometer 1108 of the example system 1100. In some implementations, the operations 1404, 1406, 1408 and 1410 may be repeatedly performed on the same swab sample or different swab samples. At 1412, data is analyzed. In some instances, the operations 1410 and 1412 may be implemented with respect to the operation 708 of the example process 700 or in another manner.

FIG. 14B is a flow chart showing an example tissue analysis process 1420. In some implementations, the example process 1420 may be an automated process used for detection and identification of molecules that are present in a tissue sample. The example process 1420 may be performed, for example, by the systems shown in FIG. 11B, or another type of system with additional or different components. The example process 1420 may include additional or different operations, including operations performed by additional or different components, and the operations may be performed in the order shown or in another order. In some cases, operations in the example process 1420 can be combined, iterated or otherwise repeated or performed in another manner.

At 1422 an analyte is formed by supplying a liquid solvent to a tissue sample. In some implementations, a control system (e.g., the control system 1006 as shown in FIG. 10) may be used to supply the liquid solvent from an external container to an internal reservoir of the sampling device (e.g., the example sampling device 300 shown in FIG. 3) that is in direct contact with the tissue sample. In some examples, the liquid solvent may stay in the internal reservoir of the sampling device for a time period (e.g., 3-15 seconds). In some implementations, the operation 1422 may be implemented with respect to the operation 406 shown in the example process 400.

At 1424, the analyte is transported. At 1426, an ionized analyte is obtained. At 1428, the ionized analyte is processed. At 1430, data is analyzed. In some implementations, the operations 1424, 1426, 1428 and 1430 in the example process 1420 may be implemented with respect to the operations 1406, 1408, 1410 and 1412 shown in the example process 1400.

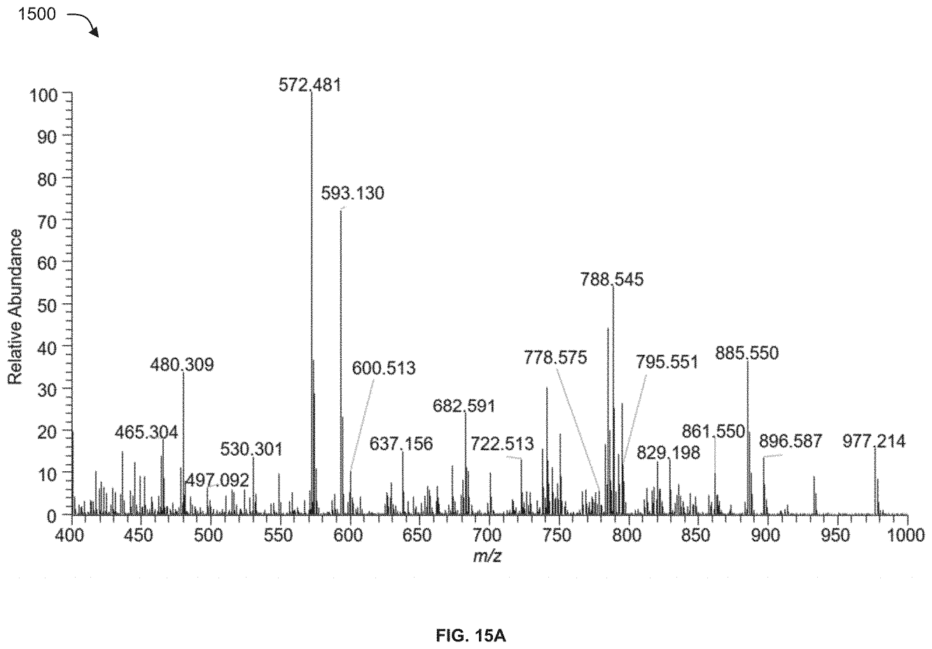

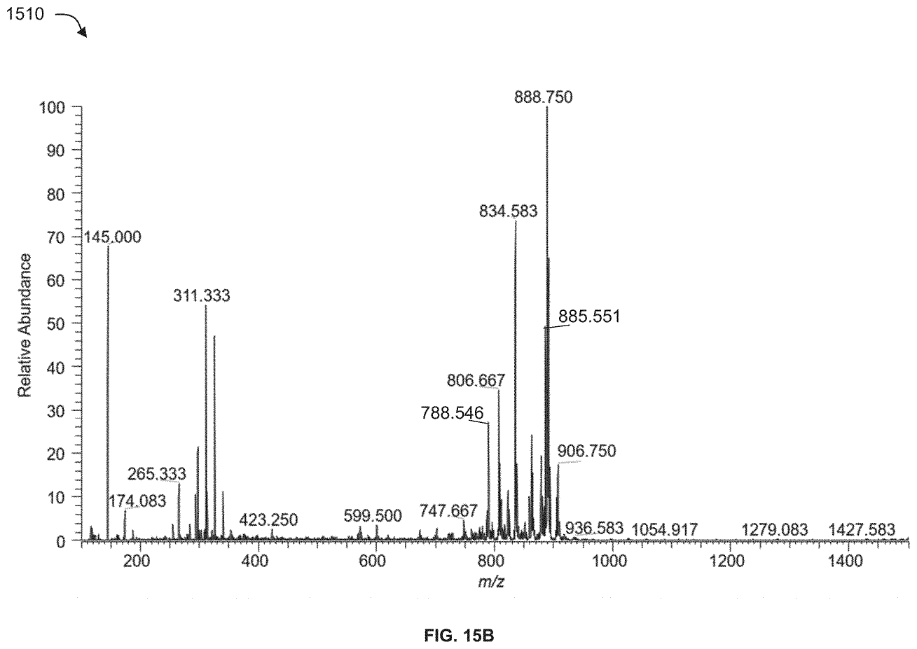

FIG. 15A is an example mass spectrum 1500 collected from a clinical nasal swab sample from a SARS-CoV-2 positive patient. The example mass spectra 1500 is obtained using the swab sample analysis system 1100 as shown in FIG. 11A with an ionization system 1200 shown in FIGS. 12A-12C. The ionization system 1200 includes an ionization source 1300 as shown in FIGS. 13A-13B. The mass spectrometer used in the swab sample analysis system 1100 is a Q Exactive HF mass spectrometer. The example mass spectra 1500 is collected according to the example process 1400 as shown in FIG. 14A. In some instances, chloroform:methanol (CHCl₃:MeOH) (1:1, v/v) or pure methanol may be used as the liquid solvent.

In some implementations, robustness of the example process 1400 using the example system 1100 can be evaluated. For example, swabs dipped in a standard solution can be used in the evaluation process. In some examples, a standard solution may contain a mixture of lipid standards including 10 micromolar (μM) of PG (m/z=773.535) and 10 μM of PE (m/z=742.540). In some instances, a ratio between the relative abundance values at m/z=773.535 and m/z=742.540 can be determined by analyzing mass spectra collected from multiple measurements (e.g., 10 times). A standard deviation of the ratios was determined as 6.45%, indicating that that the methods and systems presented here can provide reproducible and robust measurements on swab samples.

As shown in FIG. 15A, various glycerophospholipids, including phosphotidylethanolamines (PE), phosphotidylserines (PS), and phosphatidylinositols (PI), among others, were observed in the mass spectra. Several lipid species including ST 30:6 (m/z=465.304), LysoPE 18:0 (m/z=480.309), Cer 34:1 (m/z=572.481), PS 36:1 (m/z=788.545), and PI 38:4 (m/z=885.550), were observed at high relative abundances, which are consistent with the example mass spectrum 500 shown in FIG. 5A collected using the example system 200 without an ionization system. LysoPE species have been previously detected at higher abundances in cells infected with a different coronavirus, e.g., HCoV-229E. In some implementations, the methods and systems presented here using an ionization system and an ionization source can improve relative intensities of molecules and increase total numbers of molecules that can be detected. In some implementations, the methods and systems presented here using an ionization system and ionization source can detect a variety of lipid species at high relative abundances with a high reproducibility.

FIG. 15B is an example mass spectrum 1510 collected from a mouse brain tissue section. The example mass spectra 1510 is obtained using the sample analysis system 1130 shown in FIG. 11B. The mass spectrometer used in the swab sample analysis system 1130 as shown in FIG. 11B is a LTQ XL ion trap. The sample analysis system 1130 also includes an ionization system 1200 shown in FIGS. 12A-12C with an ionization source 1300 shown in FIGS. 13A-13B. The example mass spectra 1510 is collected according to the example process 1400 as shown in FIG. 14B. In some examples, CHCl₃:MeOH (1:1, v/v) or pure methanol can be used as the liquid solvent. As shown in FIG. 15B, PS 36:1 (m/z=788.546), PS 40:6 (m/z=834.583), PI 38:4 (m/z=885.551), and ST 24:1 (m/z=888.750) were detected, which are consistent with species that are commonly detected in mouse brain tissue using a variety of mass spectrometry techniques.

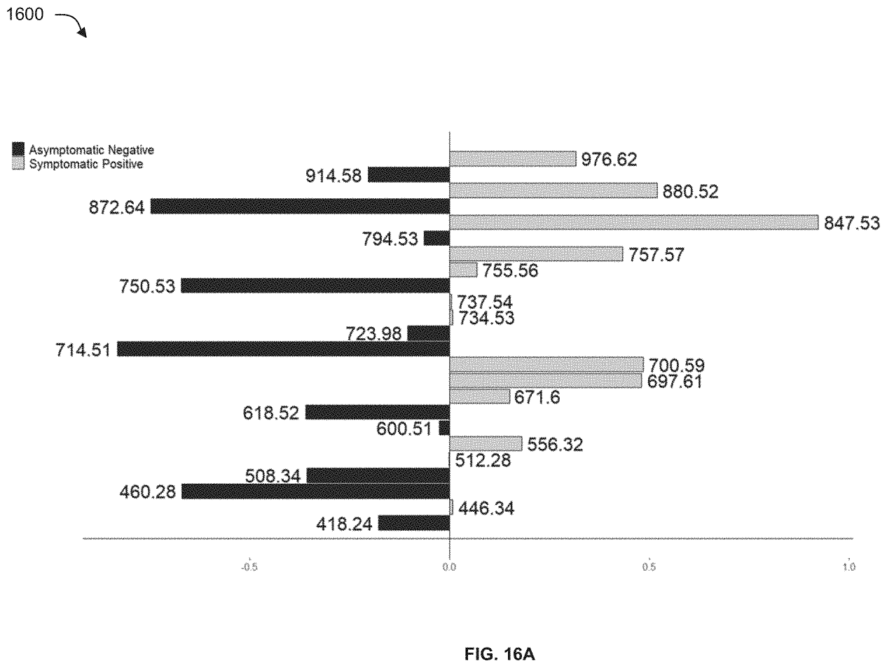

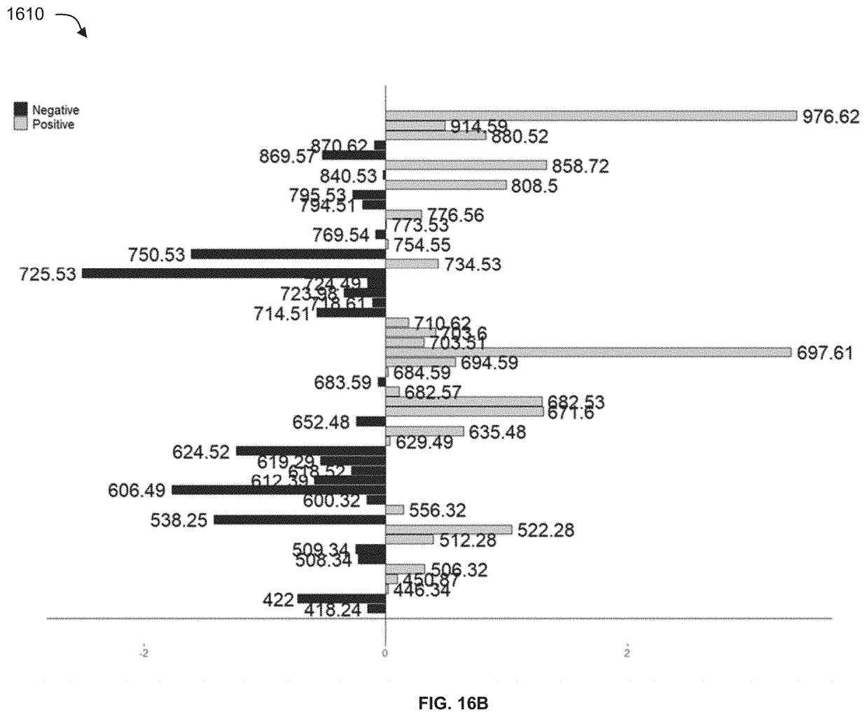

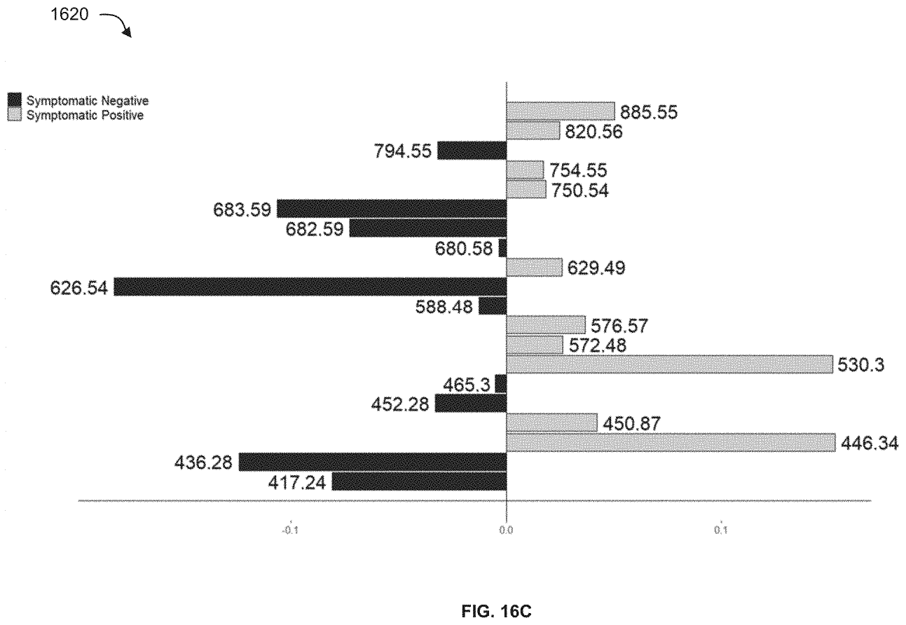

FIGS. 16A-16C are plots 1600, 1610, and 1620 showing molecular ion peaks and statistical weights associated with the molecular ion peaks in the example statistical models.

As shown in FIG. 16A, a first statistical model is trained for discriminating asymptomatic negative and symptomatic COVID-19 positive. In some instances, the molecular ion peaks and the respective statistical weights are selected by the first statistical model based on mass spectrometry profiles collected from a training set of samples including 101 asymptomatic negative samples and 44 symptomatic COVID-19 positive samples. The first statistical model yielded 91.0% overall agreement with PCR results, 88.6% sensitivity (FNR of 11.4%), and 92.1% specificity (FPR of 7.9%). The model exhibited a strong performance using LOOCV, yielding an area under the receiver operating characteristic (ROC) curve (AUC) of 0.934. A prediction probability value of 0.340 was selected as optimal threshold value for sample classification based on the ROC curve. Samples with a probability lower than 0.340 were classified as asymptomatic negative and those with a probability higher than 0.340 were classified as symptomatic COVID-19 positive.

25 predictive features, which were selected by the first statistical model, include various lipids. Molecular ion peaks which are selected by the first statistical model that are weighted toward classification of asymptomatic negative are located at m/z=418.24, 460.28, 508.34, 600.51, 618.52, 714.51, 723.98, 750.53, 794.53, 872.64, and 914.58. Molecular ion peaks which are selected by the first statistical model that are weighted toward classification of symptomatic COVID-19 positive are located at m/z=446.34, 512.28, 556.32, 671.6, 682.59, 697.61, 700.59, 734.53, 737.54, 755.56, 757.57, 847.53, 880.52, and 976.62.

Tentatively identified features weighted towards asymptomatic negative include LPE 0-18:3 [M−H]⁻ (m/z 460.28), LPE 20:0 [M−H]⁻ (m/z 508.34), Cer 36:1 [M+Cl]− (m/z 600.51), Cer 36:0 [M+Cl]−(m/z 618.52), PE 34:2 [M−H]−(m/z 714.51), and PS 42:1 [M−H]−(m/z 872.64). Tentatively identified features weighted towards symptomatic COVID-19 positive include LPC 18:1 [M+Cl]−(m/z 556.32), Cer 42:2 [M+Cl]−(m/z 682.59), Cer 44:5 [M+Cl]−(m/z 700.59), PE 34:0 [M−H]−(m/z 734.53), SM 34:1 [M+Cl]−(m/z 737.54), PA 40:1 [M−H]−(m/z 757.57), PA 48:12 [M−H]−(m/z 847.53), and PE 50:9 [M+Cl]−(m/z 976.62).

In some implementations, the statistical weights of the respective molecular ion peaks that are indicative of different samples may have different signs in the statistical model. For example, the statistical weights with negative values are indicative of asymptomatic negative and the statistical weights with positive values are indicative of symptomatic COVID-19 positive. In some implementations, the statistical weights may be configured in another manner according to the statistical model. The methods and systems presented here can be used for differentiating asymptomatic negative samples and symptomatic COVID-19 positive samples, or another application.

As shown in FIG. 16B, a second statistical model is trained for discriminating negative samples (e.g., asymptomatic, and symptomatic negative) and symptomatic COVID-19 positive samples. In some instances, the molecular ion peaks and the respective statistical weights are selected by the second statistical model based on mass spectrometry profiles collected from a training set of samples including 127 negative samples and 44 symptomatic COVID-19 positive samples. The second statistical model yielded 86.0% overall agreement with PCR results, 84.1% sensitivity (FNR of 15.9%), and 86.6% specificity (FPR of 13.4%). The model exhibited a strong performance using LOOCV, yielding an area under the receiver operating characteristic (ROC) curve (AUC) of 0.884. A prediction probability value of 0.258 was selected as optimal threshold value for sample classification based on the ROC curve. Samples with a probability lower than 0.258 were classified as negative and those with a probability higher than 0.258 were classified as symptomatic COVID-19 positive.

51 predictive features, which were selected by the second statistical model, include various lipids. Molecular ion peaks which are selected by the second statistical model that are weighted toward classification of negative are located at m/z=418.24, 422, 508.34, 509.34, 538.25, 600.32, 606.49, 612.39, 618.52, 619.29, 624.52, 652.48, 683.59, 714.51, 718.61, 723.98, 724.49, 725.53, 750.53, 769.54, 794.51, 795.53, 840.53, 869.57, and 870.62. Molecular ion peaks which are selected by the second statistical model that are weighted toward classification of symptomatic COVID-19 positive are located at m/z=446.34, 450.87, 506.32, 512.28, 522.28, 556.32, 629.49, 635.48, 671.6, 682.53, 682.57, 684.59, 694.59, 697.61, 703.51, 703.6, 710.62, 734.53, 754.55, 773.53, 776.56, 808.5, 858.72, 880.52, 914.59, and 976.62.

Tentatively identified features weighted towards negative include LPE 20:0 [M−H]− (m/z 508.34), LPE 0-26:7 [M+Cl]− (m/z 600.32), LPI 20:4 [M−H]− (m/z 619.29), PE 34:2 [M−H]− (m/z 714.51), CL 72:7 [M−2H]2−(m/z 724.49), DG 42:7 [M−H]− (m/z 725.53), PE 36:2 [M+Cl]− (m/z 794.51), and PC 38:6 [M+Cl]− (m/z 840.53). Tentatively identified features weighted towards symptomatic COVID-19 positive include LPE 20:1 [M−H]− (m/z 506.32), LPS 18:1 [M−H]− (m/z 522.28), LPC 18:1 [M+Cl]− (m/z 556.32), DG 34:1 [M+Cl]−(m/z 629.49), DG 0-36:5 [M+Cl]− (m/z 635.48), Cer 43:3 [M+Cl]− (m/z 694.59), DG 40:6 [M+Cl]− (m/z 703.51), Cer 44:2 [M+Cl]− (m/z 710.62), PE 34:0 [M−H]− (m/z 734.53), PC 0-32:0 [M+Cl]− (m/z 754.55), PG 36:2 [M−H]− (m/z 773.53), PE 0-40:6 [M−H]− (m/z 776.56), PE 0-40:8 [M+Cl]− (m/z 808.5), and PE 50:9 [M+Cl]− (m/z 976.62).

In some implementations, the statistical weights of the respective molecular ion peaks that are indicative of different samples may have different signs in the statistical model. For example, the statistical weights with negative values are indicative of negative and the statistical weights with positive values are indicative of symptomatic COVID-19 positive. In some implementations, the statistical weights may be configured in another manner according to the second statistical model. The methods and systems presented here can be used for differentiating negative samples and symptomatic COVID-19 positive samples, or another application.

As shown in FIG. 16C, a third statistical model is trained for discriminating symptomatic negative samples and symptomatic COVID-19 positive samples. In some instances, the molecular ion peaks and the respective statistical weights are selected by the third statistical model based on mass spectrometry profiles collected from a training set of samples including 26 symptomatic negative samples and 44 symptomatic COVID-19 positive samples. The third statistical model yielded 74.3% overall agreement with PCR results, 61.5% sensitivity (FNR of 38.5%), and 81.8% specificity (FPR of 18.2%). The model exhibited a strong performance using LOOCV, yielding an area under the receiver operating characteristic (ROC) curve (AUC) of 0.744. A prediction probability value of 0.514 was selected as optimal threshold value for sample classification based on the ROC curve. Samples with a probability lower than 0.514 were classified as symptomatic negative and those with a probability higher than 0.514 were classified as symptomatic COVID-19 positive.

20 predictive features, which were selected by the third statistical model, include various lipids. Molecular ion peaks which are selected by the third statistical model that are weighted toward classification of symptomatic negative are located at m/z=417.24, 436.28, 452.28, 465.3, 588.48, 626.54, 680.58, 682.59, 683.59, and 794.55. Molecular ion peaks which are selected by the third statistical model that are weighted toward classification of symptomatic COVID-19 positive are located at m/z=446.34, 450.87, 530.3, 572.48, 576.57, 629.49, 750.54, 754.55, 820.56, and 885.55.

Tentatively identified features weighted towards symptomatic negative include LPA 0-18:3 [M−H]− (m/z 417.24), LPE 0-16:1 [M−H]− (m/z 436.28), LPE 16:0 [M−H]− (m/z 452.28), Cholesterol Ester [M−H]− (m/z 465.3), Cer 34:1 [M+Cl]− (m/z 588.48), Cer 42:3 [M+Cl]− (m/z 680.58), Cer 42:2 [M+Cl]− (m/z 682.59), and PC 34:1 [M+Cl]− (m/z 794.55). Tentatively identified features weighted towards symptomatic COVID-19 positive include LPC 16:0 [M+Cl]− (m/z 530.3), Cer 34:1 [M+Cl]− (m/z 572.48), DG 34:1 [M+Cl]− (m/z 629.49), PE 0-38:5 [M−H]− (m/z 750.54), PC 0-32:0 [M+Cl]− (m/z 754.55), PC 36:2 [M+Cl]− (m/z 820.56), and PI 38:4 [M−H]− (m/z 885.55).

In some implementations, the statistical weights of the respective molecular ion peaks that are indicative of different samples may have different signs in the statistical model. For example, the statistical weights with negative values are indicative of symptomatic negative and the statistical weights with positive values are indicative of symptomatic COVID-19 positive. In some implementations, the statistical weights may be configured in another manner according to the third statistical model. The methods and systems presented here can be used for differentiating symptomatic negative samples and symptomatic COVID-19 positive samples, or another application.

Some of the subject matter and operations described in this specification can be implemented in digital electronic circuitry, or in computer software, firmware, or hardware, including the structures disclosed in this specification and their structural equivalents, or in combinations of one or more of them. Some of the subject matter described in this specification can be implemented as one or more computer programs, i.e., one or more modules of computer program instructions, encoded on a computer storage medium for execution by, or to control the operation of, data-processing apparatus. A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media.

Some of the operations described in this specification can be implemented as operations performed by a data processing apparatus on data stored on one or more computer-readable storage devices or received from other sources.

The term “data-processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an Arduino board, an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program, or in multiple coordinated files (e.g., files that store one or more modules, sub programs, or portions of code). A computer program can be deployed to be executed on one computer or on multiple computers that are located at one site or distributed across multiple sites and interconnected by a communication network.

Some of the processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

In a general aspect of what is described above, a swab sample is analyzed, for example, to test for a disease.

In a first example, an analyte is formed by mixing material from a swab sample with a solvent, and the analyze is provided to a mass spectrometer. The mass spectrometer processes the analyte to generate mass spectrometry data. The mass spectrometry data is analyzed to identify a disease marker.

Implementations of the first example may include one or more of the following features. The swab sample (e.g., the swab head containing material extracted from a subject) may be inserted into a reservoir, and the swab sample can be in direct contact with the liquid solvent and interact with the liquid solvent for a certain period of time. A specimen can be extracted from the swab head of the swab sample to form the analyte in the reservoir. The reservoir can be a reservoir in a sampling device that includes only a single reservoir, a reservoir in a cartridge that contains many such reservoirs, or a reservoir in another type of device.

Implementations of the first example may include one or more of the following features. Identifying a disease marker may include identifying a marker of whooping cough, diphtheria, influenza, a disease (e.g., SARS, MERS, or COVID-19) caused by the coronavirus family of viruses, strep throat, pneumonia, and tonsillitis. A determination of whether the disease marker is present can be used to provide a diagnosis, for example, to diagnose COVID-19 or another type of disease.

In a second example, a liquid solvent is supplied to a swab head via a first channel of a sampling device by operation of a control system. The liquid solvent interacts with the swab head to form an analyte in an internal reservoir. The analyte is transferred from the internal reservoir via a second channel of the sampling device to a mass spectrometer. The mass spectrometer performs analysis on the analyte to produce mass spectrometry data. The mass spectrometry data are analyzed to detect (e.g., to detect the presence of, or a level of) a virus (e.g., SARS-CoV-2 virus) in the analyte. The virus may be detected and identified, for example, using the mass spectrometry data and a statistical classification model for diagnosing the coronavirus disease.

Implementations of the second example may include one or more of the following features. The swab head is inserted into the internal reservoir through an opening on the sampling device. The sampling device includes a gas channel that can communicate gas (e.g., air) to the internal reservoir. The internal reservoir interfaces with the swab head, and the liquid solvent in the internal reservoir contacts the swab head. The swab head can include a sample that may or may not contain a disease marker (e.g., a marker of the SARS-CoV-2 virus), and the mass spectrometry data may be analyzed to detect the disease marker. The analysis may include detecting and identifying the disease marker for SARS-CoV-2 virus or another disease. The swab head may include a specimen collected from a swabbing procedure. The liquid solvent may include a mixture of chloroform and ethanol. The statistical classification model can be trained based on molecular features in mass spectrometry data generated by the mass spectrometer to diagnose the coronavirus disease.

In a third example, a liquid solvent is supplied to a swab head via a first channel of a sampling device by operation of a control system. The liquid solvent interacts with the swab head to form an analyte in an internal reservoir. The analyte is transferred from the internal reservoir via a second channel of the sampling device to an ionization system to form an ionized analyte. The ionized analyte is transferred to a mass spectrometer. The mass spectrometer performs analysis on the analyte to produce mass spectrometry data. The mass spectrometry data are analyzed to detect (e.g., to detect the presence of, or a level of) a virus (e.g., SARS-CoV-2 virus) in the analyte. The virus may be detected and identified, for example, using the mass spectrometry data and a statistical classification model for diagnosing the coronavirus disease.

Implementations of the third example may include one or more of the following features. The ionization system includes an electrospray ionization source. The electrospray ionization source includes a transfer capillary and can be integrated with an chamber. The transfer capillary is fluidically coupled with the sampling device to guide the analyte from the internal reservoir of the sampling device to the chamber. The chamber is under vacuum. The electrospray ionization source includes an insulating tube coaxially arranged outside a portion of the transfer capillary. The electrospray ionization source is configured to form a coaxial sheath gas between an inner wall of the insulating tube and the outer wall of the transfer capillary.

In a general aspect of what is described above, a tissue sample is analyzed, for example, to identify molecules that are present in the tissue sample.

In a fourth example, an analyte is formed by supplying a solvent to a tissue sample. An ionization system transports the analyte to an chamber under vacuum through an electrospray ionization source. The electrospray ionization source generates an ionized analyte in the chamber. A mass spectrometer processes the ionized analyte to generate mass spectrometry data.

In a fifth example, a swab head of a swab sample is inserted through an opening into an internal reservoir of a sampling device. The sampling device includes the opening, an inlet channel, an outlet channel, and the internal reservoir. The internal reservoir is in fluid communication with the inlet channel, the outlet channel, and the opening. A liquid solvent is supplied to the swab head in the internal reservoir via the inlet channel of the sampling device. The swab head is held in the liquid solvent for a period of time to form an analyte in the internal reservoir. The analyte is extracted from the internal reservoir via the outlet channel of the sampling device. The analyte is transferred to a mass spectrometer. The analyte is processed, to obtain mass spectrometry data by operation of the mass spectrometer.

Implementations of the fifth example may include one or more of the following features. Prior to inserting the swab head of the swab sample through the opening into the internal reservoir of the sampling device, a specimen from a target region of a subject is collected by inserting a clean swab into the target region. The sampling device includes a sealing element at the opening. The opening resides on a first end of a tip portion of the sampling device. After inserting the swab head of the swab sample through the opening into the internal reservoir, the opening at the tip portion is sealed by the sealing element. The liquid solvent includes sterile water. When the liquid solvent is supplied to the swab head, the sterile water is supplied to the internal reservoir; and at least a portion of the internal reservoir is filled up to allow the sterile water to interact with at least a portion of the swab head.

Implementations of the fifth example may include one or more of the following features. When the analyte is processed to obtain the mass spectrometry data, the analyte is received at an ionization system; an ionized analyte is obtained by operation of the ionization system; and the ionized analyte is subject to a mass spectrometry analysis. The ionization system includes a chamber and a vacuum source. When the analyte is received at the ionization system, the chamber is evacuated using the vacuum source to create a first pressure in the chamber. The first pressure is less than the atmospheric pressure in an environment of the sampling device. The ionization system is an electrospray ionization source. The ionization system includes a transfer capillary. When the ionized analyte is obtained, the analyte is extracted into a first end of the transfer capillary via the outlet channel; a discharge voltage is applied to a second, opposite end of the transfer capillary to form the ionized analyte, and at least a portion of the transfer capillary is heated. The second, opposite end of the transfer capillary resides in the chamber of the ionization system. The second, opposite end of the transfer capillary in the chamber is positioned in proximity to an inlet tube of the mass spectrometer. The ionization system includes a coupler. A gas flow is received on a first port of the coupler. A coaxial gas sheath is formed around the transfer capillary. The coaxial gas sheath flows from the first port to a second port of the coupler and is configured to guide the ionized analyte from the second, opposite end of the transfer capillary to the inlet tube of the mass spectrometer.

Implementations of the fifth example may include one or more of the following features. The analyte includes a liquid sample collected from the swab head. A mass spectrometry profile of the liquid sample is generated by operation of the mass spectrometer. The swab sample includes a plurality of swab samples. The inlet channel includes a plurality of inlet channels. The outlet channel includes a plurality of outlet channels. The opening includes a plurality of openings. Respective swab samples are inserted into respective internal reservoirs through respective openings. Liquid solvent is supplied to the respective swab samples in the respective internal reservoirs through respective inlet channels. Respective analytes are extracted from the respective internal reservoirs through respective outlet channels. The analytes are transferred to the mass spectrometer and processed to obtain mass spectrometry data. The mass spectrometry data is analyzed to identify whether one or more disease markers are present. When the one or more disease markers are identified, one or more disease markers of COVID-19 are identified.

In a sixth example, a swab sample analysis system includes a container, a mass spectrometer, a sampling device, and a control system. The container includes a liquid solvent. The mass spectrometer is configured to produce mass spectrometry data by processing an analyte. The sampling device includes an opening; an internal reservoir configured to receive a swab head inserted into the sampling device through the opening, and to hold the swab head in a fixed volume of the liquid solvent for a period of time to form the analyte in the sampling device; a first channel configured to communicate the liquid solvent into the internal reservoir; and a second channel configured to communicate the analyte from the internal reservoir. The control system is configured to perform operations including: supplying the liquid solvent to the internal reservoir through the first channel of the sampling device; extracting the analyte from the internal reservoir through the second channel of the sampling device; and transferring the analyte to the mass spectrometer.

Implementations of the sixth example may include one or more of the following features. The swab sample analysis system includes an ionization system configured to ionize the analyte, and the operations include transferring the analyte to the ionization system. The ionization system includes a transfer capillary configured to receive the analyte. The ionization system includes a heating element attached to the transfer capillary. The heating element is configured to heat the analyte in the transfer capillary to obtain a vaporized analyte. The ionization system is configured to ionize the vaporized analyte to produce an ionized analyte. The ionization system is an electrospray ionization system including a chamber. When the analyte is transferred to the ionization system, the analyte is sprayed into the chamber of the ionization system through a tip of a transfer capillary.

Implementations of the sixth example may include one or more of the following features. The opening resides on one end of a tip portion of the sampling device. The sampling device includes a sealing element at the opening; and the sealing element is configured to seal the opening after the swab head is inserted into the internal reservoir. The opening resides on one end of the sampling device. When the analyte is extracted from the internal reservoir through the second channel of the sampling device, air is received from an atmosphere of the sampling device to the internal reservoir through the opening. The swab sample analysis system includes one or more computer systems which are configured to analyze the mass spectrometry data to detect a substance present on a swab head. The sampling device includes a gas channel configured to supply gas to the internal reservoir. The one or more computer systems are configured to analyze the mass spectrometry data to identify whether one or more disease markers are present. When the one or more disease markers are identified, one or more disease markers of COVID-19 are identified.

In a seventh example, a system includes an external container, an electrospray ionization system, a mass spectrometer, one or more computer systems, a sampling device, and a control system. The external container includes a liquid solvent. The electrospray ionization system is configured to ionize an analyte. The mass spectrometer is configured to produce mass spectrometry data by processing the ionized analyte provided by the electrospray ionization system. One or more computer systems are configured to analyze the mass spectrometry data to detect a substance present on a sample surface. The sampling device includes an internal reservoir, a first channel, a second channel, and a third channel. The internal reservoir is configured to hold a fixed volume of the liquid solvent in direct contact with the sample surface for a period of time to form the analyte in the sampling device. The first channel is configured to communicate the liquid solvent into the internal reservoir. The second channel is configured to communicate gas into the internal reservoir. The third channel is configured to communicate the analyte from the internal reservoir. The control system is configured to perform operations including: supplying the liquid solvent to the internal reservoir through the first channel of the sampling device; extracting the analyte from the internal reservoir through the third channel of the sampling device; and transferring the analyte to the electrospray ionization system by applying a first pressure in the electrospray ionization system. The first pressure is less than the atmospheric pressure in an environment of the sampling device.

Implementations of the seventh example may include one or more of the following features. The electrospray ionization system includes a transfer capillary configured to receive the analyte. When the analyte is transferred, the analyte is transferred from the internal reservoir to the transfer capillary. The electrospray ionization system includes a heating element attached to the transfer capillary. When the analyte is ionized, the analyte is heated by operation of the heating element in the transfer capillary to obtain a vaporized analyte; and the vaporized analyte is ionized to produce the ionized analyte. The electrospray ionization system includes a chamber. When the analyte is transferred to the electrospray ionization system, the analyte is aspirated into the chamber of the electrospray ionization system through a tip of the transfer capillary. The transfer capillary is electrically conductive. When the analyte is ionized, a discharge voltage is applied on the transfer capillary to obtain the ionized analyte. The electrospray ionization system includes a chamber. The mass spectrometer includes an inlet tube residing in proximity to a tip of the transfer capillary in the chamber. The operations include applying a second pressure on the inlet tube. The second pressure on the inlet tube is less than the first pressure.

Implementations of the seventh example may include one or more of the following features. The electrospray ionization system includes a chamber coupled to a vacuum source. When the first pressure is applied, the chamber is evacuated to the first pressure by operation of the vacuum source. The system includes a first transfer tube and a second transfer tube. The first transfer tube communicates the liquid solvent from the external container to the first channel; and the second transfer tube communicates the analyte from the sampling device to the electrospray ionization system. The second channel includes an open end that receives air from an atmosphere of the sampling device. The fixed volume is defined by the volume of the internal reservoir. The sampling device is a handheld sampling device. The sample surface includes a surface of a biological tissue.

In an eighth example, a liquid solvent is supplied through a first channel of a sampling device to an internal reservoir of the sampling device. A fixed volume of the liquid solvent is held in the internal reservoir in direct contact with a sample surface for a period of time to form an analyte in the sampling device. Gas is supplied to the internal reservoir of the sampling device through a second channel of the sampling device. The analyte is extracted from the internal reservoir through a third channel of the sampling device and transferred to an electrospray ionization system by applying a first pressure in the electrospray ionization system. The first pressure is less than the atmospheric pressure in an environment of the sampling device. By operation of the electrospray ionization system, the analyte from the internal reservoir is ionized. By operation of a mass spectrometer, mass spectrometry data is produced by processing the ionized analyte from the electrospray ionization system. The mass spectrometry data is analyzed to detect a substance present at the sample surface.

Implementations of the eighth example may include one or more of the following features. The electrospray ionization system includes a chamber coupled to a vacuum source. When the first pressure is applied, the chamber is evacuated to the first pressure by operation of the vacuum source. The electrospray ionization system includes a transfer capillary that receives the analyte. When the analyte is transferred, the analyte is transferred from the sampling device to the transfer capillary. When the analyte is ionized, the analyte is heated in the transfer capillary to obtain a vaporized analyte; and the vaporized analyte is ionized to produce the ionized analyte. The electrospray ionization system includes a chamber. When the analyte is transferred to the electrospray ionization system, the analyte is sprayed into the chamber of the electrospray ionization system through a tip of the transfer capillary by forming a gas sheath around at least a portion of the transfer capillary. The transfer capillary is electrically conductive. When the analyte is ionized, a discharge voltage is applied on the transfer capillary to obtain the ionized analyte. The electrospray ionization system includes a chamber. The mass spectrometer includes an inlet tube residing in proximity to a tip of the transfer capillary in the chamber. A second pressure is applied on the inlet tube, wherein the second pressure on the inlet tube is less than the first pressure. The ionized analyte is provided to the mass spectrometer by collecting the ionized analyte through the inlet tube.

Implementations of the eighth example may include one or more of the following features. The first channel receives the liquid solvent from the external container through a first transfer tube. The analyte is transferred from the sampling device to the electrospray ionization system through a second transfer tube. The second channel receives the gas through an open port that receives air from an atmosphere of the sampling device. The fixed volume is defined by the volume of the internal reservoir. The sample surface includes a surface of a biological tissue.

While this specification contains many details, these should not be understood as limitations on the scope of what may be claimed, but rather as descriptions of features specific to particular examples. Certain features that are described in this specification or shown in the drawings in the context of separate implementations can also be combined. Conversely, various features that are described or shown in the context of a single implementation can also be implemented in multiple implementations separately or in any suitable sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single product or packaged into multiple products.

A number of embodiments have been described. Nevertheless, it will be understood that various modifications can be made. Accordingly, other embodiments are within the scope of the present disclosure.

1-23. (canceled)

24. A system comprising:
an external container comprising a liquid solvent;
an ionization system configured to ionize an analyte;
a mass spectrometer configured to produce mass spectrometry data by processing the ionized analyte provided by the ionization system;
one or more computer systems configured to analyze the mass spectrometry data to detect a substance present on a sample surface;
a sampling device comprising:
an internal reservoir configured to hold a fixed volume of the liquid solvent in direct contact with the sample surface for a period of time to form the analyte in the sampling device;
a first channel configured to communicate the liquid solvent into the internal reservoir;
a second channel configured to communicate gas into the internal reservoir; and
a third channel configured to communicate the analyte from the internal reservoir; and
a control system configured to perform operations comprising:
supplying the liquid solvent to the internal reservoir through the first channel of the sampling device;
extracting the analyte from the internal reservoir through the third channel of the sampling device; and
transferring the analyte to the ionization system by applying a first pressure in the ionization system, the first pressure being less than the atmospheric pressure in an environment of the sampling device.

25. The system of claim 24, wherein:
the ionization system comprises a transfer capillary configured to receive the analyte, and
transferring the analyte comprises transferring the analyte from the internal reservoir to the transfer capillary.

26. The system of claim 25, wherein
the ionization system comprises a heating element attached to the transfer capillary, and ionizing the analyte comprises:
by operation of the heating element, heating the analyte in the transfer capillary to obtain a vaporized analyte; and
ionizing the vaporized analyte to produce the ionized analyte.

27. The system of claim 25, wherein the ionization system comprises a chamber, and transferring the analyte to the ionization system comprises aspirating the analyte into the chamber of the ionization system through a tip of the transfer capillary.

28. The system of claim 25, wherein the ionization system is an electrospray ionization system, the transfer capillary is electrically conductive, and ionizing the analyte comprises applying a discharge voltage on the transfer capillary to obtain the ionized analyte.

29. The system of claim 25, wherein the ionization system comprises a chamber, the mass spectrometer comprises an inlet tube residing in proximity to a tip of the transfer capillary in the chamber, the operations comprise applying a second pressure on the inlet tube, and the second pressure on the inlet tube is less than the first pressure.

30. The system of claim 24, wherein the ionization system comprises a chamber coupled to a vacuum source, and applying the first pressure comprises evacuating the chamber to the first pressure by operation of the vacuum source.

31. The system of claim 24, comprising:
a first transfer tube that communicates the liquid solvent from the external container to the first channel; and
a second transfer tube that communicates the analyte from the sampling device to the ionization system.

32. The system of claim 24, wherein the second channel comprises an open end that receives air from an atmosphere of the sampling device.

33. The system of claim 24, wherein the fixed volume is defined by the volume of the internal reservoir.

34. The system of claim 24, wherein the sampling device is a handheld sampling device.

35. The system of claim 24, wherein the sample surface comprises a surface of a biological tissue.

36. A method comprising:
supplying a liquid solvent through a first channel of a sampling device to an internal reservoir of the sampling device;
holding a fixed volume of the liquid solvent in the internal reservoir in direct contact with a sample surface for a period of time to form an analyte in the sampling device;
supplying gas to the internal reservoir of the sampling device through a second channel of the sampling device;
extracting the analyte from the internal reservoir through a third channel of the sampling device;
transferring the analyte to an ionization system by applying a first pressure in the ionization system, the first pressure being less than the atmospheric pressure in an environment of the sampling device;
ionizing, by operation of the ionization system, the analyte from the internal reservoir;
by operation of a mass spectrometer, producing mass spectrometry data by processing the ionized analyte from the ionization system; and
analyzing the mass spectrometry data to detect a substance present at the sample surface.

37. The method of claim 36, wherein the ionization system comprises a chamber coupled to a vacuum source, and applying the first pressure comprises evacuating the chamber to the first pressure by operation of the vacuum source.

38. The method of claim 36, wherein:
the ionization system comprises a transfer capillary that receives the analyte, and
transferring the analyte comprises transferring the analyte from the sampling device to the transfer capillary.

39. The method of claim 38, wherein ionizing the analyte comprises:
heating the analyte in the transfer capillary to obtain a vaporized analyte; and
ionizing the vaporized analyte to produce the ionized analyte.

40. The method of claim 38, wherein the ionization system comprises a chamber, and transferring the analyte to the ionization system comprises spraying the analyte into the chamber of the ionization system through a tip of the transfer capillary by forming a gas sheath around at least a portion of the transfer capillary.

41. The method of claim 38, wherein the ionization system is an electrospray ionization system, the transfer capillary is electrically conductive, and ionizing the analyte comprises applying a discharge voltage on the transfer capillary to obtain the ionized analyte.

42. The method of claim 38, wherein the ionization system comprises a chamber, the mass spectrometer comprises an inlet tube residing in proximity to the tip of the transfer capillary in the chamber, and the method comprises:
applying a second pressure on the inlet tube, wherein the second pressure on the inlet tube is less than the first pressure; and
providing the ionized analyte to the mass spectrometer by collecting the ionized analyte through the inlet tube.

43. The method of claim 36, wherein the first channel receives the liquid solvent from the external container through a first transfer tube, the analyte is transferred from the sampling device to the ionization system through a second transfer tube, and the second channel receives the gas through an open port that receives air from an atmosphere of the sampling device.

44. The method of claim 36, wherein the fixed volume is defined by the volume of the internal reservoir.

45. The method of claim 36, wherein the sample surface comprises a surface of a biological tissue.
